(19) United States
(12) Patent Application Publication
(10) Pub. No.: US 2025/0009787 A1
(43) Pub. Date: Jan. 9, 2025

(54) Nitric Oxide Containing Foam Formulation for Topical Medical Use

(71) Applicant: NOxy Health Products, Inc., Anaconda, MT (US)

(72) Inventors: C. Michael Miller, Anaconda, MT (US); David A. Bell, Farmington, UT (US)

(21) Appl. No.: 18/758,691

(22) Filed: Jun. 28, 2024

Related U.S. Application Data
(60) Provisional application No. 63/511,243, filed on Jun. 30, 2023.

(51) Int. Cl. A61K 33/00; A61K 9/00; A61K 9/12; A61K 47/02; A61K 47/12; A61K 47/18; A61P 31/02

(52) U.S. Cl. CPC A61K 33/00; A61K 9/0014; A61K 9/122; A61K 47/02; A61K 47/12; A61K 47/186; A61P 31/02

(57) ABSTRACT

Disclosed herein are topical nitric oxide (NO) formulations containing a surfactant and methods that deliver NO to treat skin wounds, burns, infections, site of inflammations, or other skin conditions requiring treatment, as well as to eradicate and degrade biofilms on the skin surface. The NO formulations can be modified to prolong the time of NO exposure by modulating the surfactant concentration.

### CROSS-REFERENCE TO RELATED APPLICATIONS

This application claims priority to U.S. Provisional Application Ser. No. 63/511,243, filed Jun. 30, 2023, the entire contents of which are incorporated by reference herein.

### BACKGROUND

Nitric oxide (NO) is a known antimicrobial, vasodilator, and signaling molecule that can be used to treat humans and animals suffering from a range of medical afflictions, particularly those associated with the skin. For example, in 2004, Miller reported the use of NO gas to treat leg abscesses. According to the report, gaseous NO was applied to the leg of a patient using a “single patient use” plastic boot. The boot was equipped with a delivery system that administered 200 ppm NO gas for an average of 8.1 hours. After 14 consecutive nights of treatment, the lesion was significantly reduced in size. Miller, J Cutan Med Surg, 2004 July-August; 8(4):233-8. doi: 10.1007/s10227-004-0106-8. In US 2005/0191372, Miller further discloses NO gas bathing units, including an inflatable bag-like bathing units in the shape of a boot or mitten or glove that can be placed over a patient's foot or hand, respectively. Other reports relating to the use of NO to treat skin conditions abound.

Miller illustrates the real-world challenges of using NO gas for wound treatment. NO gas is reported to be toxic at higher concentration (greater than exposures of 200 ppm). In Miller's bathing units, it is impossible to control the directionality of NO gas diffusion to just the site on the skin requiring treatment. Treatment effects are thus due to serendipitous absorption of NO. As a result, Miller's method and device requires long treatment times and high concentrations of NO gas, increasing the risk of toxic exposure.

To avoid the issues posed by NO gas treatment, therapeutic media such as creams and ointments have been developed that generate NO in situ from the reaction of nitrite with acid. These creams and ointments are two-part formulations. The first formulation contains the nitrite, and the second formulation contains the acid. When the two formulations are mixed, for instance on skin that requires treatment, the acid and nitrite react to form NO gas, which then diffuses to the skin. Zhu H, Ka B, Murad F, World J. Surg. 2007, 31, 624. Zhu H, Wei X, Bian K, Murad F, J. Burn Care Res. 2008, 29, 804.

Researchers have also utilized foams for NO delivery to skin requiring treatment. In U.S. Pat. No. 10,751,365 Miller, Hill and Bell disclose a two-part liquid formulation that is to provide NO to the skin. The first formulation contains nitrite and a surfactant, and the second formulation contains acid and a surfactant. The two formulations can be agitated to produce foams. When the two foamed formulations are mixed, the acid and nitrite react to form NO which is present as NO-containing bubbles in the foam, which then can be placed on skin requiring treatment.

However, challenges remain in using NO-containing foam as a delivery medium, particularly for treating infections on the skin that are caused by microbial pathogens including bacteria, viruses, and fungi, as well as for treating inflammatory dermal conditions.

### SUMMARY

These and other needs are met by the present invention, which harnesses the antimicrobial, vasodilator, and cell signaling properties of NO to treat a variety of topical skin infections and conditions using a NO foam formulation. We have surprisingly found that modifying the NO foam formulations by adjusting the surfactant concentration makes it possible to modify the foam collapse rate and thus the rate of NO delivery to a site on the skin that requires treatment, so that longer or shorter treatment times can be achieved as needed. Prolonged exposure times for the NO topical foam formulation means prolonged times for NO to exert its therapeutic effect and/or antimicrobial effect, for the foam to serve as a protective covering, and for the foam to exert its moistening quality.

Thus, in one aspect, what is provided is an NO topical foam formulation, comprising NO, water, and a surfactant, wherein the concentration of the surfactant is 0.1% weight/weight (w/w) to 50% w/w. A further embodiment of this aspect is a formulation comprising additional components, which are described herein below.

In another aspect, what is provided is a method of treating an infection, site of inflammation, or skin condition with NO in a patient requiring such treatment, comprising contacting the skin with the NO topical foam formulation as disclosed herein for an extended or specified period of time. In this aspect, the site requiring treatment is a skin wound, burn, infection, site of inflammation, or other condition requiring treatment.

In another aspect, what is provided is a method of treating, inhibiting, or eradicating an infection on the skin of a patient requiring such treatment, comprising:
applying an NO topical foam formulation foam as disclosed herein to the skin requiring treatment;
allowing the NO topical foam formulation to remain on the skin for a period of time to allow the NO to treat the infection; and
allowing the NO topical foam formulation to remain on the skin for a period of time to allow the NO to be absorbed;
wherein the infection is caused by a bacteria, a virus, or a fungus; and
wherein the therapeutic effect is an antimicrobial effect, vasodilatory effect, or wound healing effect.

In another aspect, what is provided is a method of disrupting or degrading a biofilm on the skin of a patient requiring such treatment and providing a therapeutic effect, comprising: applying a NO topical foam formulation foam as disclosed here to the skin requiring treatment;
allowing the NO topical foam formulation to remain on the skin for a period of time to allow the NO to treat the infection; and
allowing the NO topical foam formulation to remain on the skin for a period of time to allow the NO to be absorbed;
wherein the biofilm encases bacteria, a virus, or a fungus; and
wherein the therapeutic effect is an antimicrobial effect, vasodilatory effect, or wound healing effect.

In another aspect, what is provided is a method of prolonging a therapeutic effect of the NO topical foam formulation as disclosed herein, the method comprising increasing the surfactant concentration of the NO topical foam formulation and decreasing the rate of NO delivery, wherein the therapeutic effect is an antimicrobial effect, a vasodilatory effect, or a wound healing effect.

In another aspect, what is provided is a therapeutic covering for an application site on the skin of a patient requiring such treatment, wherein the covering is the NO topical formulation foam as disclosed herein, wherein the covering protects the application site on the skin or tissue from ambient interference and environmental contaminants such as air- or waterborne pathogens.

In another aspect, what is provided is a method of:
(i) treating, inhibiting, or eradicating an infection on a topical site of a patient requiring such treatment, or of
(ii) disrupting, degrading, or removing a biofilm on the topical site of a patient requiring such treatment, or of
(iii) reducing or removing the bioburden on the topical site of a patient requiring such treatment, or of
(iv) removing or loosening foreign material from a topical site; or of
(v) disinfecting a topical site of a human or animal comprising:
   applying the NO topical foam formulation foam as recited in claims 1-13 to the topical site; and
   allowing the NO topical foam formulation to remain on the topical site for a period of time to allow the NO to be absorbed to provide a therapeutic effect;
   wherein the therapeutic effect is an antimicrobial effect, vasodilatory effect, or healing effect.

In another aspect, what is provided is a method of:
(i) treating, inhibiting, or eradicating an infection on a topical site of a patient requiring such treatment, or of
(ii) disrupting, degrading, or removing a biofilm on the topical site of a patient requiring such treatment, or of
(iii) reducing or removing the bioburden on the topical site of a patient requiring such treatment, or of
(iv) removing or loosening foreign material from a topical site; or of
(v) disinfecting a topical site of a human or animal comprising:
   applying the NO topical foam formulation foam as recited in claims 1-13 to the topical site; and
   allowing the NO topical foam formulation to remain on the topical site for a period of time to allow the NO to treat, inhibit, or eradicate the infection or to disrupt, degrade, or remove the biofilm, or to reduce or remove the bioburden, or to remove or loosen the foreign material, or to disinfect;
   wherein the infection, biofilm, or bioburden is caused by a bacteria, a virus, or a fungus.

In another aspect, what is provided is the use of the topical foam formulation disclosed herein to treat a topical site on a patient requiring treatment, comprising contacting the site with a NO topical foam formulation comprising NO, water, and a surfactant, wherein the concentration of the surfactant is 0.1 weight/weight (w/w) to 50% w/w.

In another aspect, what is provided is the use of the topical foam formulation disclosed herein in the manufacture of a medicament to treat a topical site on a patient requiring treatment, comprising contacting the site with a NO topical foam formulation comprising NO, water, and a surfactant, wherein the concentration of the surfactant is 0.1 weight/weight (w/w) to 50% w/w.

In another aspect, what is provided is the use of the NO topical foam formulation disclosed herein to treat an infection, site of inflammation, or skin condition in a patient requiring such treatment, comprising contacting the skin with the NO topical foam formulation.

In another aspect, what is provided is the use of the NO topical foam formulation disclosed herein in the manufacture of a medicament to treat an infection, site of inflammation, or skin condition in a patient requiring such treatment, comprising contacting the skin with the NO topical foam formulation.

In another aspect, what is provided is the use of the NO topical foam formulation disclosed herein to prolong a therapeutic effect of the NO topical foam formulation comprising increasing the surfactant concentration of the NO topical foam formulation and decreasing the rate of NO delivery, wherein the therapeutic effect is an antimicrobial effect, a vasodilatory effect, or a wound healing effect.

In another aspect, what is provided is the use of the NO topical foam formulation disclosed herein in the manufacture of a medicament to prolong a therapeutic effect of the NO topical foam formulation comprising increasing the surfactant concentration of the NO topical foam formulation and decreasing the rate of NO delivery, wherein the therapeutic effect is an antimicrobial effect, a vasodilatory effect, or a wound healing effect.

In another aspect, what is provided is a therapeutic covering for an application site on the skin of a patient requiring such treatment, wherein the covering is the NO topical formulation disclosed herein, wherein the covering protects the application site on the skin or tissue from ambient interference and environmental contaminants such as air- or waterborne pathogens.

### BRIEF DESCRIPTIONOF THE FIGURES

FIG. 1 depicts the process of nitrite foam delivery of NO gas as described herein

FIG. 2 depicts the Drip Flow Reactor (DFR) that was employed to create biofilms.

FIG. 3 is an example of a foam pump.

FIG. 4 is the Log CFU/cm² bar chart of P. aeruginosa tests (* indicates statistical difference from control; **indicates statistical difference from NO treatment.

FIG. 5 is the Log CFU/cm² bar chart of A. baumannii tests (* indicates statistical difference from control; **indicates statistical difference from NO treatment.

FIG. 6 is the Log CFU/cm² bar chart of S. aureus tests (* indicates statistical difference from control; **indicates statistical difference from NO treatment.

FIG. 7 is the Log CFU/cm² bar chart of C. albicans tests (* indicates statistical difference from control; **indicates statistical difference from NO treatment.

FIG. 8 is the Log CFU/cm² bar chart of S. epidermidis tests (* indicates statistical difference from control; **indicates statistical difference from NO treatment.

FIG. 9 is the Log CFU/cm² bar chart of P. mirabilis (* indicates statistical difference from control; **indicates statistical difference from NO treatment.

FIG. 10 summarizes NOx release rate for three foams containing different concentrations of surfactant.

FIG. 11 summarizes NOx release rate for three foams containing different concentrations of surfactant.

FIG. 12 shows the collapse rate of foams relative to surfactant concentration.

### DETAILED DESCRIPTION

### Definitions

As used herein, “infection” refers to an infection on the topical site of a human or animal subject that is caused by bacteria, viruses, or fungi. The site is typically a skin site.

As used herein, “skin” means the covering of a human or animal's body consisting of the epidermis (outermost layer), the dermis (fibrous layer that supports and strengthens the epidermis), and the subcutis (a subcutaneous layer of fat beneath the dermis). “Skin” also means unbroken skin and broken skin, where broken skin may include breaks in epidermis, dermis, and subcutis, down to muscular tissue and even down to bone, as in a deep cut, puncture, or other wound.

As used herein, “skin infection” means an infection in the skin caused by bacteria, viruses, or fungi. A “bacterial infection of the skin” means an infection caused by planktonic bacteria or bacteria in a biofilm, and can include infections caused by P. aeruginosa, A. baumannii, S. aureus, S. epidermidis, P. mirabilis, Propionibacterium acnes, (P. acnes), Cutibacterium acnes, or Malassezia spp, or as otherwise disclosed herein. Other bacteria-causing skin infections include Staph aureus, Strep pyogenes, Strep agalactiae, Staph dysgalctiae, Staph anginosos, Staph intermedius, Staph constellatus, Entero faecais, E. coli, Klebisella pneumonia, Enterobacter cloacae, among others.

For example, bacterial skin infections can take the form of carbuncles, ecthyma, erythrasma, folliculitis, furuncles, impetigo, lymphadenitis, skin abscesses, cellulitis, erysipelas, lymphangitis, necrotizing skin infections, and wound infections. Aerobic bacteria, such as S. aureus, S. epidermidis, and Pseudomonas aeruginosa are often found on the surface of chronic wounds, while anaerobic bacteria are found in deeper tissue, such as Bacteroides spp., Fusobacterium spp., Peptostreptococcus spp., as well as Clostridium spp, among others.

As used herein, a “biofilm” is a densely packed aggregate of bacterial, viral or fungal microorganisms encased in a matrix of extracellular polymeric substance. Within a biofilm, microorganisms such as bacteria are embedded in a glycocalyx. The glycocalyx is a self-produced matrix of extracellular polymeric substance (EPS) which consists of polysaccharides, lipids, proteins, and extracellular DNA. The EPS is considered to be the hallmark of biofilms.

The formation of a biofilm begins with the attachment of free-floating microorganisms to a surface followed by microcolony formation. During surface colonization, the organisms are able to communicate using quorum sensing (QS) products such as N-acyl homoserine lactone. A polysaccharide matrix encloses microbes and they mature. This is followed by the final stage of dispersion.

Biofilms offer unique advantages to the organisms including protection from host defenses, metabolic cooperation, increased virulence, differential gene expression, and increased resistance to antimicrobials. In vitro studies show that the bacteria in biofilms are 50-500 times more resistant to antibiotics than their planktonic forms. Several factors including physical barrier, altered growth and metabolism, increased mutation, gene transfer, phenotype switching, persisters or spore-like forms, increased efflux pumps, and production of enzymes contribute to antimicrobial tolerance.

Biofilms cannot be easily visualized in skin biopsies with routine light microscopy and require special techniques such as electron microscopy, epifluorescence microscopy, peptide nucleic acid-fluorescence in situ hybridization, cryo-scanning, electron microscopy, or confocal laser scanning microscopy. Biofilms are associated with various pathological conditions in humans such as cystic fibrosis, colonization of indwelling medical devices, and dental plaque formation involved in caries and periodontitis. Biofilms are also implicated in chronic wounds, Hidradenitis suppurativa, atopic dermatitis, candidiasis, acne, onychomycosis, dermal filler granuloma, miliaria, impetigo, Pemphigus foliaceus, rosacea, among other conditions.

Biofilms pose a challenge to the clinician due to the limitation of routine diagnostic culture techniques for their detection and their resistance to conventional antimicrobial therapy due to their persistent and chronic nature. Although various novel treatment options are available, they show varying degrees of efficacy and the eradication of biofilm in cutaneous diseases remains difficult. Hence, better understanding of their molecular biology, pathogenesis, and role in various diseases can help in the development of potential therapeutic strategies against biofilms in the future.

As used herein, a “viral skin infection” means an infection caused by a virus, and includes an infection caused by varicella zoster virus (chicken pox), herpes zoster virus (shingles), the pox virus that causes molluscum contagiosum, and human herpes virus 6 (HHV-6) (roseola), rubeola virus (measles), parvovirus (fifth disease), Epstein Barr virus (mononucleosis), among others.

As used herein, a “fungal skin infection” means an infection caused by a fungi, and includes infections caused by Candida albicans (yeast), as well as other fungal infections, including Tinea corporis (ringworm of the body), Tinea capitis (ringworm of the scalp), Tinea cruris (jock itch), Tinea versicolor, Tinea pedis (athlete's foot), onychomytosis, and fungal skin rashes, among others.

As used herein, “disinfection,” for instance, as used in the phrase “skin disinfection,” refers to the process of applying a disinfectant to reduce levels of microorganisms on the skin. The NO topical foam formulation disclosed herein can be used as a disinfectant.

As used herein, “inflammatory skin conditions” include a range of conditions that arise from a variety of causes. They present as skin eruptions, and rashes. Inflammatory skin conditions include eczema, psoriasis, acne, folliculitis, and allergic urticarial, atopic dermatitis, and contact dermatitis, among others.

“Bioburden” means all of the dimensions of wound microbiology believed to be important in the development of wound infection. These dimensions include microbial load, microbial diversity, and the presence of pathogenic organisms.

As used herein, a “wound” is a break or opening in the skin that can be infected by bacteria, viruses, or fungi. Non-limiting examples of wounds include cuts, lacerations, gashes, tears, scrapes, punctures, abrasions, scratches, ulcers, burns (chemical, electric, radiation, and thermal), and lesions.

As used herein, “skin ailment” and “skin condition” are used interchangeably and refer to an ailment or condition that causes damage to skin (which includes the epidermis, dermis, and subcutis), and possibly the underlying tissue. Non-limiting examples of skin ailments include acne, dermatitis, eczema, herpes, hives, shingles, and skin abscesses, among others.

As used herein, coco betaine and cocamidopropyl betaine are different compounds and are examples of cationic biobased surfactants produced from natural raw materials such as coconut oil.

As used herein, “dermatological agent” and “dermatological topical agent” can be used interchangeably and refer to therapeutic agents as well as topical excipients that can be applied directly on the skin to treat skin conditions. They deliver medicines to treat or prevent dermatologic conditions or are used as excipients for topical dermatologic products or in wound dressings. As is known to the skilled person, many topical dermatologic agents and excipients described herein may serve more than one purpose, and it is not intended for the topical dermatologic agents and excipients to be limited to a single purpose.

Types of dermatological agents include topical agents for miscellaneous use, topical acne agents, topical anesthetics, topical anti-infectives, topical anti-rosacea agents, topical antibiotics, topical antifungals, topical antihistamines, topical antineoplastics, topical antipsoriatics, topical antivirals, topical astringents, topical debriding agents, topical depigmenting agents, topical emollients, topical keratolytics, topical non-steroidal anti-inflammatories, topical photochemotherapeutics, topical rubefacients, topical steroids, topical steroids with anti-infectives, topical cleansers, topical steroids with anti-infectives, ingredients that help in angiogenesis, anorectal agents, skin protectants, odor managers, as well as moisture managers, topical enzymes, ingredients in wound dressings, and topical antiseptics. Topical excipients include humectants, emulsifiers, preservatives, adhesives, thickeners, and others as described herein.

Dermatologic agents are used to treat a variety of skin conditions and may have multiple biological effects. Non-limiting examples of topical dermatologic agents used to treat skin conditions that are described herein include aluminum chloride hexahydrate, crisaborole (CAS Reg. No. 906673-24-3), eflornithine, tacrolimus, pimecrolimus, minoxidil, glycopyrronium, menthol, ionic zinc, coal tar, capsaicin, selenium sulfide, bimatoprost, diphenhydramine, sodium hyaluronate, salicylic acid, Vitamin E, bexarotene, mequinol/tretinoin, becaplermin, dexpanthenol, and alitretinoin.

Topical acne agents include antiseptic washes that contain ingredients to gently cleanse the skin; and creams, lotions, or gels that exfoliate the skin, inhibit bacterial growth, speed up skin cell renewal or decrease the formation of comedones. Non-limiting examples of topical acne agents include adapalene, benzoyl peroxide, clindamycin, dapsone, tretinoin, azelaic acid, tazarotene, salicylic acid, clascoterone, erythromycin, and resorcinol.

Topical anesthetics are medicines that numb and reduce the sensation of pain in the area to which they are applied. Non-limiting examples of topical anesthetics include lidocaine, pramoxine, phenol, benzocaine, dibucaine, xylocaine, tetracaine, prilocaine, benzocaine, wintergreen (CAS Reg. No. 68917-75-9), and dyclonine.

Topical anti-infective agents act by either killing or inhibiting the spread of infectious agents. They include antibiotics, antibacterial (antimicrobial), antifungal and antiviral agents. Non-limiting examples of topical anti-infectives include docosanol, ivermectin, imiquimod, hydrogen peroxide, crotamiton, spinosad, cadexomer iodine, malathion, piperonyl butoxide, pyrethrins, permethrin, sinecatechins, abametapir, acetic acid, iodoquinol, nitrofurazone, and chloroxine, mupirocin (CAS Reg. No. 12650-69-0), polyhexanide (polyaminopropyl biguanide), crystal violet (CAS Reg. No. 548-62-9), gentian violet (CAS Reg. No. 548-62-9), lemon, sodium hypochlorite, and povidone-iodine solution, polymyxin (CAS Reg. No. 1405-20-5), cavacrol (CAS Reg. No. 499-75-2), sandalwood oil (CAS Reg. No. 8006-87-9), thymol (CAS Reg. No. 89-83-8), decanoic acid, Methylene blue (CAS Reg. No. 61-73-4), hypochlorous acid, sorbic acid, acetic acid, grapefruit extract (CAS 90045-43-5), and Medicinal honey.

Topical anti-rosacea agents are used for the treatment of inflammatory papules, pustules and erythema of rosacea. Non-limiting examples of anti-rosacea agents include ivermectin, brimonidine, oxymetazoline, and azelaic acid.

Topical antibiotics are medicines that destroy or inhibit the growth of susceptible bacteria. Non-limiting examples of topical antibiotics include sulfacetamide sodium/sulfur, retapamulin, ozenoxacin, erythromycin, polymyxin b, mafenide, gentamicin, and mupirocin.

Topical antifungals are products that treat fungal infections caused by dermatophytes, yeasts, or mold. Non-limiting examples of topical antifungals include efinaconazole, tavaborole, ketoconazole, terbinafine, undecylenic acid, nystatin, econazole, ciclopirox, naftifine, oxiconazole, clotrimazole, sertaconazole, tolnaftate, butenafine, luliconazole, miconazole, and sulconazole.

Topical antihistamines are products that have been manufactured for use on the skin, in the nose, or in the eye. They contain antihistamines which are medicines that block histamine release from histamine-1 receptors and are used to treat the symptoms of an allergic reaction such as edema (swelling), itch, inflammation (redness), sneezing, or a runny nose or watery eye. Non-limiting examples of topical antihistamines include doxepin and diphenhydramine.

Topical antineoplastics work by different mechanisms to prevent the development and spread of neoplastic cells that characterize cancers such as melanoma. Non-limiting examples of topical antineoplastics include imiquimod, fluorouracil, ingenol, ruxolitinib, tirbanibulin, and mechlorethamine.

Topical antipsoriatics are agents, which are applied on the skin surface to treat psoriasis. Non-limiting examples of topical antipsoriatics include betamethasone, calcipotriene, tazarotene, and halobetasol.

Topical antiviral agents are applied locally to treat viral infections. Non-limiting examples of topical antivirals include penciclovir and acyclovir.

Topical astringents are agents that cause skin cells or mucus membranes to contract or shrink, by precipitating proteins from their surface. When applied topically they dry, harden and protect the skin. A non-limiting example of a topical astringent is witch hazel.

Topical debriding agents are chemicals that are used locally to clean an open wound by removing foreign material and dead tissue, so that the wound heals without increased risk of infection. This makes the healing faster. Non-limiting examples of topical debriding agents include peru balsam (CAS Reg. No. 8007-00-9) combined with castor oil and trypsin, and anacaulase.

Topical depigmenting agents work in different ways to inhibit melanogenesis (the pigmentation pathway by which cells produce melanin). Some agents cause reversible depigmentation and some cause irreversible depigmentation. These agents are applied on the skin, on the affected area to treat hyperpigmentation. Non-limiting examples of topical depigmenting agents include fluocinolone combined with hydroquinone and tretinoin, and monobenzoin.

Topical emollients, or moisturizers, contain ingredients that soothe and soften the skin. Non-limiting examples of topical emollients include salicylic acid/urea, ammonium lactate, urea, petrolatum, lanolin, mineral oil, cetyl dimethicone copolyol, cyclomethicone, palm glyceride(s), panthenol (CAS Reg. No. 81-13-0), and aloe vera.

Topical keratolytics are agents that are applied on the skin to soften the keratin. They loosen and assist exfoliation of the skin cells. Keratolytics also help the skin to bind moisture and are useful in treating dry skin conditions. They are used to treat psoriasis, acne, warts, corns and other forms of keratosis. Non-limiting examples of topical keratolytics include podofilox (CAS Reg. No. 518-28-5), salicylic acid, hydrocolloids, and trichloroacetic acid.

Topical non-steroidal anti-inflammatories (often abbreviated to NSAIDs) are creams, gels, rubs, solutions or sprays that contain a nonsteroidal anti-inflammatory agent and are designed to be applied directly to the skin overlying a painful joint or area of bone. They are used to relieve pain and to treat symptoms of arthritis such as inflammation, swelling, and stiffness. Topical NSAIDs may also be used in the treatment of actinic keratosis (a precancerous patch of thick, scaly or crusted skin). Non-limiting examples of topical NSAIDs include diclofenac, capsaicin/diclofenac, and diclofenac/lidocaine.

Topical photochemotherapeutics make skin more sensitive to light. They work by causing a reaction with light that can destroy certain types of diseased skin cells. They may be used in the treatment of vitiligo or actinic keratosis in combination with light treatment. Non-limiting examples of topical photochemotherapeutics include aminolevulinic acid, methoxsalen, methylamino levulinate, and aminolevulinic acid.

Topical rubefacients cause irritation and reddening of the skin, due to increased blood flow. They are used in the treatment of pain in various musculoskeletal conditions. Non-limiting examples of topical rubefacients include methyl salicylate, camphor, menthol, and capsaicin/menthol.

Topical steroids contain corticosteroids (often abbreviated to steroids) which are designed to be applied externally to the scalp or the skin, depending on the condition being treated. Corticosteroids control inflammation by mimicking naturally occurring corticosteroid hormones produced by adrenal glands, which are two small glands that sit on top of kidneys. In addition to reducing inflammation (redness and swelling) in the area that they are applied, topical corticosteroids also suppress the immune response, reduce cell turnover, and constrict (narrow) blood vessels. Non-limiting examples of topical steroids include mometasone, clobetasol, triamcinolone, fluocinonide, flurandrenolide, clocortolone, halobetasol, desoximetasone, desonide, betamethasone, halcinonide, fluocinolone, prednicarbate, diflorasone, triamcinolone, fluticasone, and alclometasone.

Topical antiseptics are used to reduce the microbial count and reduce the risk of infections on the skin. Non-limiting examples of topical antiseptics include alcohol (ethanol and isopropanol), benzethonium chloride, benzalkonium chloride (BAC), camphorated metacresol, eucalyptol 0.091%, hexylresorcinol, hydrogen peroxide topical solution, iodine tincture, iodine topical solution, menthol, methylbenzethonium chloride, methyl salicylate, phenol, povidone-iodine, menthol, citric acid, sodium tetraborate, benzoic acid, polyaminopropyl biguanide, polyhexylmethylene biguanide, and thymol. BAC represents a mixture of N,N-dimethyl alkyl

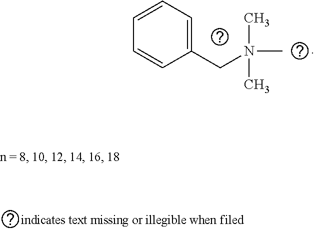

amines, which conform generally to the formula.

“Topical dermatologic agent” also includes agents that can aid topically in angiogenesis such as trans-cinnamnaldehyde (CAS Reg. No. 14371-10-9); and anorectal agents such as oak extract (CAS Reg. No. 68917-11-3).

“Topical dermatologic agent” also includes skin protectants such as listed in the following table.

| Skin Protectant | CAS Reg. No. |
|---|---|
| Allantoin | 97-59-6 |
| Aluminum sulfate | 10043-0103 |
| Octadecahydrate | 7784-31-8 |
| Lanolin (hydrous) | 8006-54-0 |
| Petrolatum (petroleum jelly) | 8009-03-8 |
| Konjac flour | 37220-17-0 |
| Isopropyl sorbate | 55584-26-4 |
| Keratin | 68238-35-7 |
| Palmitamide MEA | 629-54-9 |
| Zirconium oxide | 1314-23-4 |
| Sucrose octasulfate/ | 54182-58-0 |
| Aluminum hydrochloride | |
| Glycerin | 56-81-5 |
| Ascorbic acid | 50-81-7 |
| Fruit extract | |
| Glycrrhetinic acid | 471-53-4 |
| (Licorice extract) | |
| Lavender | 8000-28-0 |
| Meadowsweet extract | 7732-18-5 |
| | 56-81-5 |
| | 84775-57-5 |
| | 122-99-6 |
| Myrtillus extract | 84082-34-8 |
| Oat glucan | 9041-22-9 |
| | 9051-97-2 |
| Soy protein | 9010-10-0 |
| Triglycerol | 20411-31-8 |

“Topical dermatologic agent” also includes adhesives that can be used to attach wound dressings to the skin. Examples include hydrogenated rosin (CAS Reg. No. 64365-17-9).

“Topical dermatologic agent” also includes topical cleansers such as aqueous saline, and cocoamphodiacetate (CAS Reg. No. 68650-39-5).

“Topical dermatologic agent” also includes humectants, which are substances that attract water, that can be used in wound dressings and include such substances as butylene glycol, propylene glycol, betaines, ethoxydiglycol, pentlyene glycol, acetamide MEA (CAS Reg. No. 142-26-7), lactic acid, and sodium lactate.

“Topical dermatologic agent” also includes substances for odor management in wound dressings such as sodium copper chlorophyllin (CAS Reg. No. 65963-40-8).

“Topical dermatologic agent” also includes preservatives such as diazolidinyl urea, DMDM hydantoin (CAS Reg. No. 6440-58-0), hydrochloric acid, maltodextrin, phenoxyethanol, phosphoric acid, potassium sorbate, sodium benzoate, sodium citrate, sodium metabisulfite, sodium sulfate, Germaben® II (CAS Reg. No. 57-55-6, 78491-02-8), and Quaternium-15 (CAS Reg. 4080-31-3, 51229-78-8 (cis isomer)).

“Topical dermatologic agent” also includes topical emulsifiers such as sucrose laurate, tartaric acid, triethanolamine, Tara gum (CAS Reg. No. 39300-88-4), ceteareth-10 phosphate (CAS Reg. No. 106233-09-4), triethanolamine salicylate, and diethanolamine cetyl phosphate.

Topical thickening agents include xanthan gum, among others.

“Topical dermatologic agent” also includes agents for moisture management in wound dressings such as calcium carbonate, calcium alginate, gelling fiber from chitosan, polyvinyl alcohol fibers, carboxymethyl cellulose fibers, and hydrocolloids.

“Topical dermatologic agent” also includes topical enzymes that can be used topically, such as glucose oxidase, anacaulase, and lactoperoxidase.

Miscellaneous dermatologic agents include manganese chloride, magnesium sulfate, wheat extract, bisabolol (chamomile oil, CAS Reg. No. 23089-26-1), Camellia sinensis (leaf extract, CAS Reg. No. 84650-60-2), Centella asiatic extract (CAS Reg. No. 84696-21-9), blueberry extract (CAS Reg. No. 84082-34-8), grape seed extract (CAS Reg. No. 84929-27-1), sarcosine, Ringer's lactate (CAS Reg. No. 8022-63-7), piroctone olamine (CAS Reg. No. 68890-66-4), sulfur dioxide, honey including Manuka honey, hydrogels, and temesteine, recombinant human platelet derived growth factor, Karaya gum (cas Reg. No. 9000-36-6), carboxymethyl cellulose, silicone gels containing polydimethylsiloxane, hydroxyl- and dihydroxysiloxane, vitamin a, d, and e, ascorbyl tetraisopalmitate, and alkyl siloxane resin. Miscellaneous dermatological agents also include ingredients in wound dressings such as ferric chloride hexahydrate, calcium salts, thrombin, potassium ferrate, and RADA-16 peptide.

As used herein, “therapeutic effect” means treatment of any kind, the results of which are judged to be useful or favorable.

All amounts provided herein are given as weight percents based on the total weight (i.e., 100 weight percent) of a given solution.

### Embodiments

As noted above, the present invention harnesses the antimicrobial, vasodilator, and signaling properties of NO to treat a variety of topical skin infections and conditions using a NO foam formulation. In one aspect, what is provided is an NO topical foam formulation, comprising NO, water, and a surfactant, wherein the concentration of the surfactant is 0.1% weight/weight (w/w) to 50% w/w. In another aspect, what is provided is a method of treating an infection, site of inflammation, or skin condition with NO in a patient requiring such treatment, comprising contacting the skin with the NO topical foam formulation as disclosed herein for an extended or specified period of time.

In these and other aspects, the patient requiring treatment has an infection, skin wound, burn, site of inflammation, or other condition requiring treatment. In these and other aspects, the infection is caused by microbial pathogens, including bacteria, viruses, or fungi. The composition and method employs NO-laden, micro-bubble foam to create an air-tight barrier that effectively isolates the skin to be treated and that minimizes escape of NO to the surrounding environment. Overall, the specified NO foam may be considered as a temporary dilute dispersion of bubbles, distorted to form polyhedral cells with relatively thin, flat walls, wherein the polyhedra are almost regular dodecahedra with Plateau borders. The foam, comprised of a plethora of thin-filmed, micro (to nano) sized bubbles, forms a transitional and impermeable bubble system, held together by electro-static and surface tensioning forces.

Specifically, in one embodiment, the topical foam is comprised of interdependent volumes of acidic and nitrite ion solutions that, when combined, form a foam containing NO gas bubbles created at a pre-determined reaction between the acidic and nitrite solutes. The NO gas bubbles are internally isolated within the foam. The foam clusters around the NO gas bubbles creating a barrier that blocks the diffusion of NO gas from the foam to the surrounding environment. The foam thus provides a therapeutic covering for, and isolation of, an application site on the skin, ensuring it is free from ambient interference and environmental contaminants such as air- or waterborne pathogens. In effect, the foam is a molecular bandage that efficiently protects and allows for the efficient administration of NO to the site in need of treatment.

Once applied to the site, the foam forms a barrier overlay the site that traps NO bubbles and prevents them from escaping to the surrounding environment, and that naturally compels NO bubbles that are next to the surface to rupture due to shock, vibration, temperature gradients, texture, or to surface tension modifiers, thus liberating NO gas at sites requiring treatment. Ultimately the foam provides a medium for prolonged moistening and administering NO to the site, thereby preparing the application site for sterile bandaging.

NO bubble clusters contained within the foam are optimally suited to cover irregular surfaces that are typical of surface wounds, cuts, abrasions, tears, abscesses, and the like. The result is that sites requiring treatment can be completely covered with a layer of therapeutic NO bubbles which in turn expose every facet of wound geography to molecular NO gas. Each bubble is of a predetermined and uniform density. When taken together, the bubbles provide an air-tight network over and above the application site, which, apart from the NO gas itself, limit exogenous micro- to nanoscopic elements from either entering, leaving, or disrupting the barrier. These bubbles are designed to be relatively durable to house the NO gas that is generated in situ, protecting the gas from premature dispersion, as well as carrying, transporting, and offloading individual NO gas payloads on cue to sites on the skin that require treatment.

These isolating steps are preparatory to a systematic flooding of a site on the skin requiring treatment with NO, thereby effectively and efficiently promoting in situ orthogonal diffusion of the gas into skin tissue. Adjusting the surfactant concentration in the NO containing foam formulation allows additional control over foam collapse and NO bubble rupture, prolonging the ability of the foam to deliver NO gas to a site requiring treatment. Fine-tuning surfactant concentration enables longer exposure times, ensuring even greater efficacy for the NO gas contained in bubbles in the foam, in disrupting biofilms on the surface and treating infections, skin conditions, and dermal inflammation, as well as to aide in wound healing. Moreover, since NO is a signaling molecule and a known vasodilator, increasing the likelihood of NO absorption through the skin can be harnessed to treat other pathologies in the body, in addition to those associated with the outer layers of the skin.

The NO containing foams can be prepared as herein described. The components include at least one substance with an acidic pH (that is, a pH of from 1 to 6.9), one or more surfactants, and a source of NO.

In one embodiment, the at least one substance with an acidic pH is at least protic acid selected from the group consisting of citric acid, lactic acid, salicylic acid, phosphoric acid, ascorbic acid, hydrochloric acid, acetic acid, hyaluronic acid, hypochlorous acid, gluconic acid, aspartic acid, formic acid, fumaric acid, galacturonic acid, malonic acid, formic acid, acetoglutaric acid, gluconic acid, glutamic acid, butyric acid, glutaric, butyric acid, shikimic acid, propionic acid, pyruvic glyoxylic acid, 2-hydroxybutyric acid, α-hydroxyglutaric acid, isocitric acid, lactic acid, malic acid, methylmalonic acid, quinic acid, succinic acid, tartaric acid, and oxalic acid, or a combination thereof. In a further embodiment, the at least one protic acid is citric acid.

Honey, including Manuka honey, acacia honey, clover honey, orange blossom honey, various wildflower honeys, as well as a variety of other honeys is the source for a combination of protic acids, including gluconic acid, aspartic acid, citric, acetic, formic, fumaric, galacturonic, malonic, formic, acetoglutaric, gluconic, glutamic, butyric, glutaric, butyric, shikimic, propionic, pyruvic glyoxylic, 2-hydroxybutyric, α-hydroxyglutaric, isocitric, lactic, malic, methylmalonic, quinic, succinic, tartaric, oxalic and others. In a further embodiment, the at least one protic acid is selected from the group consisting of citric acid, ascorbic acid, hypochlorous acid, and honey, or a combination thereof. In a further embodiment, the at least one protic acid is Manuka honey. Manuka honey is a honey that is produced from the nectar of the Manuka tree, Leptospermum scoparium that has antibacterial and anti-inflammatory qualities that make it useful for treating a variety of skin conditions.

In one embodiment, the surfactant is a pharmaceutically acceptable cationic surfactant that can be used topically on the skin. In a further embodiment, the surfactants are selected from cetyl trimethyl ammonium bromide, cetrimonium bromide, dodecylbenzenesulfonic acid, cetylpyridinium chloride, stearalkonium chloride, polyquaternium-7, cocamidopropyl betaine, coco betaine, lauryl dimethyl ammonium chloride, polyquaternium-10, behentrimonium chloride, and cetrimonium chloride, or combinations thereof. In one embodiment, the surfactant is cocamidopropyl betaine. In a further embodiment, the surfactant is coco betaine. In one embodiment, the NO comes from a source of NO. In a further embodiment, the NO comes from a source comprising one or more of a NO gas, nitrite salt, NONOate, NO-polymer, nano-crystalline NO, NO-metal, NO silica particles, nitrate esters, nitroprusside, nitrosamine, L-arginine, L-citrulline, nitrosothiols, NO synthase or synthase upregulators.

In a further embodiment, the NO source is a nitrite salt selected from the group consisting of sodium nitrite, calcium nitrite, potassium nitrite, and ammonium nitrite or a combination thereof. The NO source is typically sodium nitrite or potassium nitrite.

The formulations employed to make the NO topical foam formulation comprise two formulations, both of which are batch scalable. A first formulation may comprise approximately 100 mL of water, and an acidic solution of for example, 6 g of citric acid, and 3 g of cationic surfactant. A second formulation may comprise approximately 100 mL of water, 10 g of sodium nitrite, and 1 g of cationic surfactant.

Alternatively, a first formulation may comprise approximately 100 mL of water, 5 g of lactic acid, 8 g of citric acid, and 3 g of cationic surfactant. A second formulation may comprise approximately 100 mL of water, 10 g of sodium nitrite, 2 g of sodium bicarbonate, and 1 g of cationic surfactant.

Alternatively, the first formulation may comprise approximately 100 mL of water, 5 g of lactic acid, 6 g of citric acid, and 6 g of cationic surfactant. A second formulation may comprise approximately 100 mL of water, 10 g of sodium nitrite, and 2 g of cationic surfactant.

Alternatively, a first formulation may comprise approximately 100 mL of water, 5 g of lactic acid, 8 g of citric acid, and 6 g of cationic surfactant. A second solution may comprise approximately 100 mL of water, 10 g of sodium nitrite, 2 g of sodium bicarbonate, and 1 g of cationic surfactant.

Alternatively, a first formulation may comprise approximately 50-99 g of water, 0.1 to 50 g of a cationic surfactant, and 9 to 15 g of citric acid. A second solution may comprise approximately 50 to 99 g of water, 0.1 to 50 g of cationic surfactant, and 17 to 23 g of sodium nitrite.

Alternatively, a first formulation may comprise approximately 80 to 99 g of water, 1 to 20 g of a cationic surfactant, and 10 to 14 g of citric acid. A second solution may comprise approximately 80 to 99 g of water, 1 to 20 g of cationic surfactant, and 18 to 22 g of sodium nitrite. In this and other embodiments, the cationic surfactant is coco betaine.

Alternatively, a first formulation may comprise approximately 96 to 100 g of water, 8 to 12 g of a cationic surfactant, and 11 to 13 g of citric acid. A second solution may comprise approximately 96 to 100 g of water, 2 to 5 g of cationic surfactant, and 19 to 21 g of sodium nitrite.

Alternatively, a first formulation may comprise approximately 99 to 100 g of water, 10.5 to 11.5 g of a cationic surfactant, and 11.5 to 12.5 g of citric acid. A second solution may comprise approximately 99 to 100 g of water, 3 to 4 g of cationic surfactant, and 19 to 20 g of sodium nitrite.

In a further embodiment, the acid concentration (w/w %) of the first solution may be about 0.1 to 30.0 percent. In a further embodiment, the acid concentration (w/w %) of the first solution may be about 1 to 20 percent. In a further embodiment, the acid concentration (w/w %) of the of the first solution may be about 2 to 10 percent. In a further embodiment, the acid concentration (w/w %) of the first solution may be about 3 to 15 percent. In a further embodiment, the acid concentration (w/w %) of the of the first solution may be about 5 to 10 percent.

In a further embodiment, the acid concentration (w/w %) of the combined first solution and second solutions may be about 0.1 to 15.0 percent. In a further embodiment, the acid concentration (w/w %) of the combined first solution and second solutions may be about 1 to 10 percent. In a further embodiment, the acid concentration (w/w %) of the combined first solution and second solutions may be about 2 to 5 percent. In a further embodiment, the acid concentration (w/w %) of the combined first solution and second solutions may be about 3 to 8 percent. In a further embodiment, the acid concentration (w/w %) of the combined first solution and second solutions may be about 4 to 7 percent.

In a further embodiment, the nitrite concentration (w/w %) of the of the second solution may be about 0.1 to 30.0 percent. In a further embodiment, the nitrite concentration (w/w %) of the second solution may be about 1 to 28 percent. In a further embodiment, the nitrite concentration (w/w %) of the second solution may be about 2 to 25 percent. In a further embodiment, the nitrite concentration (w/w %) of the second solution may be about 3 to 20 percent. In a further embodiment, the nitrite concentration (w/w %) of the second solution may be about 4 to 19 percent. In a further embodiment, the nitrite concentration (w/w %) of the second solution may be about 14 to 18 percent.

In a further embodiment, the nitrite concentration (w/w %) of the of the combined first and second solutions may be about 0.1 to 15.0 percent. In a further embodiment, the nitrite concentration (w/w %) of the combined first solution and second solutions may be about 1 to 14 percent. In a further embodiment, the nitrite concentration (w/w %) of the combined first solution and second solutions may be about 2 to 12 percent. In a further embodiment, the nitrite concentration (w/w %) of the combined first solution and second solutions may be about 3 to 11 percent. In a further embodiment, the nitrite concentration (w/w %) of the combined first solution and second solutions may be about 4 to 10 percent. In a further embodiment, the nitrite concentration (w/w %) of the combined first solution and second solutions may be about 5 to 10 percent.

As noted above, in these formulations, increasing the surfactant concentration can lead to more robust foams that enhance NO exposure times. Thus, in one embodiment, the total surfactant concentration (w/w %) of the combined first solution and second solutions may be about 0.1 to 50.0 percent. In another embodiment, the total surfactant concentration (w/w %) of the combined first solution and second solutions may be about 0.1 to 30.0 percent. In another embodiment, the total surfactant concentration (w/w %) of the combined first solution and second solutions may be increased from about 1.0 to 2.0 percent to about 2.1 to 50.0 percent. In another embodiment, the total surfactant concentration (w/w %) of the combined first solution and second solutions may be increased from about 1.0 to 2.0 percent to about 2.1 to 30.0 percent. In another embodiment, the total surfactant concentration (w/w %) of the combined first solution and second solutions may be increased from about 1.0 to 2.0 percent to about 2.1 to 10.0 percent. In another embodiment, the total surfactant concentration (w/w %) of the combined first solution and second solutions may be increased from about 1.0 to 2.0 percent to about 2.1 to 6.0 percent.

In further embodiments, the formulations may be as provided in Tables 1.1-1.3, wherein the total w/w percents total 100 percent.

TABLE 1.1

| Formulation | Conc. (w/w %) |
|---|---|
| Solution A | |
| Deionized Water | 75-95% |
| Cationic Surfactant | 1.0-3.0% |
| Acid | 8-12% |
| Solution B | |
| Deionized Water | 75-95% |
| Cationic Surfactant | 0.5-1.5% |
| Nitrite | 15-20% |

TABLE 1.2

| Formulation | Conc. (w/w %) |
|---|---|
| Solution A | |
| Deionized Water | 75-95% |
| Cationic Surfactant | 3.1-7.0% |
| Acid | 8-12% |
| Solution B | |
| Deionized Water | 75-95% |
| Cationic Surfactant | 1.6-2.0% |
| Nitrite | 15-20% |

TABLE 1.3

| Formulation | Conc. (w/w %) |
|---|---|
| Solution A | |
| Deionized Water | 75-95% |
| Cationic Surfactant | 7.1-10% |
| Citric Acid | 8-12% |
| Solution B | |
| Water | 75-95% |
| Cationic Surfactant | 2.1-40% |
| Nitrite | 15-20% |

In these and other embodiments, the surfactant is coco betaine, the acid is citric acid, and the nitrite is sodium nitrite.

### Other Components

In an additional aspect, the NO topical foam formulation of the invention further comprises one or more additional topical dermatological agents, wherein the one or more additional topical dermatological agents does one or more of modifying the treatment profile of the foam, modifying the physical properties of the foam, modifying the chemical properties of the foam, or modifying the surface properties of the application site. Since the NO topical foam formulations disclosed herein have prolonged exposure times, they can also prolong the exposure times for other topical dermatological agents.

In these and other embodiments, the topical dermatologic agent is selected from the agents described herein. In these and other embodiments, the topical dermatologic agent is a therapeutic dermatologic agent or excipients used in wound dressings selected from the group consisting of agents to treat acne, topical anesthetics, topical anti-infectives including antibiotic, antimicrobials, and antivirals agents, topical anti-roasacea agents, topical antifungal agents, topical antihistamines, topical astringents, topical debriding agents, topical depigmenting agents, topical emollients, topical keratolytics, topical non-steroidal anti-inflammatory agents, topical rubefacient agents, topical steroid agents, topical antiseptic agents, topical agents that aid in angiogenesis, topical skin protectants, adhesives, topical skin cleansers, topical humectants, odor management agents as well as perfumes and fragrance oils, preservatives, emulsifiers, agents for moisture management, enzymes that can be used topically, excipients that can be used in topical skin compositions as well as wound dressings, as well as antineoplastics, antipsoriatics, photochemotherapeutics, topical steroids with anti-infectives, anorectal and thickeners.

In one embodiment, the topical dermatologic agent is selected from the group consisting of topical acne agents, topical anesthetics, topical anti-infectives, topical anti-rosacea agents, topical antibiotics, topical antifungals, topical antihistamines, topical antineoplastics, topical antipsoriatics, topical antivirals, topical astringents, topical debriding agents, topical, depigmenting agents, topical emollients, topical keratolytics, topical non-steroidal anti-inflammatories, topical photochemotherapeutics, topical rubefacients, topical steroids, topical steroids with anti-infectives, and topical antiseptics, or a combination thereof. Examples of various topical dermatologic agents are provided in the definitions section of this application.

In another embodiment, the topical dermatologic composition disclosed herein comprises a topical dermatologic antiseptic for first aid use to treat wounds including scrapes, cuts and burns. In this and other embodiments, the topical dematological antiseptic is selected from the group consisting of alcohol (ethanol and isopropanol), benzethonium chloride, benzalkonium chloride (BAC), camphorated metacresol, eucalyptol 0.091%, hexylresorcinol, hydrogen peroxide topical solution, iodine tincture, iodine topical solution, menthol, methylbenzethonium chloride, methyl salicylate, phenol, povidone-iodine, menthol, citric acid, sodium tetraborate, benzoic acid, polyaminopropyl biguanide, polyhexylmethylene biguanide, and thymol. BAC represents a mixture of N,N-dimethyl alkyl amines, which conform generally to the formula:

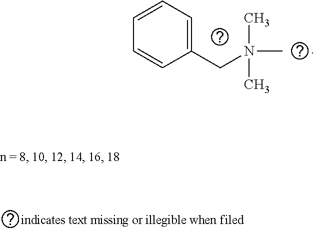

In another embodiments, the topical dermatologic antiseptic is selected from the group consisting of ethanol, isopropanol, benzethonium chloride, benzalkonium chloride (BAC), camphorated metacresol, eucalyptol 0.091%, hexylresorcinol hydrogen peroxide topical solution, iodine tincture, iodine topical solution, menthol, hypochlorous acid, methylbenzethonium chloride, methyl salicylate, phenol, povidone-iodine, and thymol.

In another embodiment, two or more topical dermatologic agents are present in the first or second solutions. In one embodiment, one topical dermatologic agent is present in one of the solutions and another topical dermatologic agent is present in the other solution. In another embodiment, both topical dermatologic agents are present in one of the solutions and the other solution does not include one or both of the topical dermatologic agents. In another embodiment, both topical dermatologic agents are present in both solutions.

In another embodiment, three or more topical dermatologic agents are present in the first or second solutions. In one embodiment, one topical dermatologic agent is present in one solution, and the other two topical dermatologic agents are present in the other solution. In another embodiment, all three topical dermatologic agents are present in one of the solutions and the other solution does not include the topical dermatologic agents. In another embodiment, all three topical dermatologic agents are present in both solutions.

In these and other embodiments, the first, second, and third topical dermatologic agents are selected from the group consisting of topical acne agents, topical anesthetics, topical anti-infectives, topical anti-rosacea agents, topical antibiotics, topical antifungals, topical antihistamines, topical antineoplastics, topical antipsoriatics, topical antivirals, topical astringents, topical debriding agents, topical depigmenting agents, topical emollients, topical keratolytics, topical non-steroidal anti-inflammatories, topical photochemotherapeutics, topical rubefacients, topical steroids, topical steroids with anti-infectives, and topical antiseptics, or a combination thereof. Examples of various topical dermatologic agents are provided in the definitions section of this application.

In these and the embodiments, the topical dermatologic agent is present based on the total weight of either the first solution, second solution, or both the first and second solutions, of 0.01 to 5 weight percent.

### Methods of Foam Formation

The foams disclosed herein can be formed in a number of ways known to the skilled person. For example, the first and second solutions can be agitated by whipping or shaking to each form a foam, and then these resultant foams can be mixed together using a stirring rod, spatula, or other common device. For example, the first and second solutions are available in first and second plastic bottles that can be shaken by hand to produce foams. The two foams are mixed, typically again by hand.

When a portion of the first and second solutions are mixed, a resultant foam is produced that contains NO which can be readily applied to a surface on a human or an animal. The resultant foam is a carrier for the in situ generation of NO due to the reaction of sodium nitrite with acid, according to the reaction sequence shown below.

2NO₂⁻+2H⁺→N₂O₃+H₂O Eq. 1

2N₂O₃→N₂O₂+NO Eq. 2

The use of a foam as a carrier provides a number of benefits. The foam carrier may effectively provide a barrier that helps to keep the NO contained near the skin, to promote topical treatment of heal cuts and scrapes, as well as to disrupting biofilms on the surface, as well as treating skin infections and inflammatory skin conditions, and to promote the absorption of NO through the skin. Although not bound by theory, a bubble containing NO gas may burst near the skin due to shock vibrations, irregularities in the surface of the skin, temperature, or another perturbation, allowing the NO to be absorbed transdermally. The foam may be placed on the skin and then allowed to collapse as opposed to being rubbed in.

The foam carrier may provide a relatively easy process for metering the amount of NO generated and put in contact with cuts, scrapes, burns, infections, and sites of skin inflammation. The foam carrier may also provide a method that uses less media as compared to other media. The resulting foams can be evaluated using a number of tests and can be tailored to meet various therapeutic needs.

In a further aspect, what is provided is a method of prolonging the protection, and anti-infective properties of the NO topical foam formulation by creating an impermeable barrier that covers the skin requiring treatment, comprising modifying one or a combination of parameters selected from the group consisting of changing the surfactant, changing the concentration of the surfactant or modifying the foam delivery rate of NO bubbles.

The lifetime of the foam can be expressed in terms of the bubble collapse rate. The foams of the present invention may have a bubble collapse rate of one second to ten minutes to one hour and up to 72 hours.

### Treatment Method

As noted above, the NO topical foam formulation can be used on the skin of animals and humans to heal cuts and scrapes, to treat infections, and to disrupt biofilms on the skin, as well as to treat inflammatory skin conditions. In one embodiment, the method comprises:
providing the first and second aqueous solutions as recited herein;
dispensing a portion of the first aqueous solution;
dispensing a portion of the second aqueous solution;
mixing the portion of the first aqueous solution and the portion of the second aqueous solution in a manner that produces a resultant foam comprising NO; and
applying the resultant foam to the site of the skin condition.

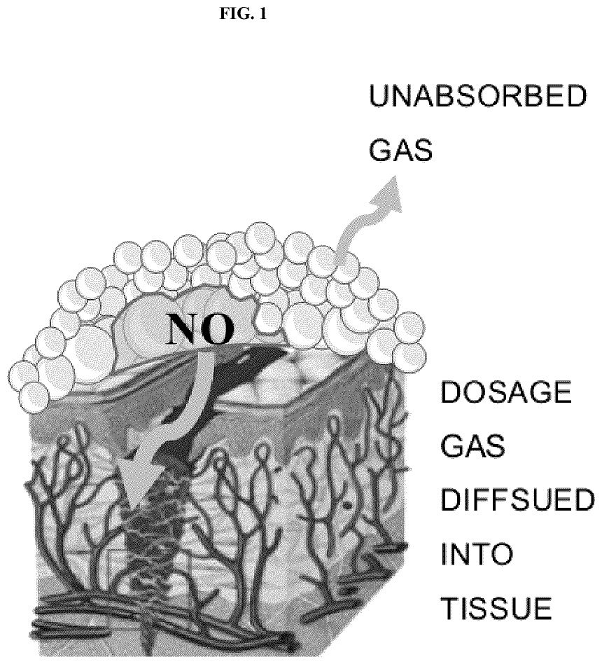

As noted previously, the first and second aqueous solutions are available in first and second plastic bottles that can be shaken by hand to produce foams. A portion of the first foam is dispensed and then mixed with a portion of the second foam which is dispensed. The two foams are mixed, typically again by hand, to produce a resultant foam carrier for the in situ generation of NO as described previously. FIG. 1 depicts the process of nitrite foam delivery of NO gas as described herein. The resultant medium may be allowed to remain on the portion of skin for a period of time so that the resultant foam collapses on the skin, thus delivering NO to the site of a wound, an infection, or the site of inflammation, or other skin irritation.

By modulating the surfactant concentration of the NO topical foam formulation, the collapse of the foam and thus NO exposure times can be modulated. For example, increasing the surfactant concentration of the NO topical foam formulation can make the foam last longer, thus increasing NO exposure times. Thus, exposure times can be increased by 10 seconds to 72 hours by increasing the surfactant concentration of the NO topical foam formulation, or from 25 to 175 percent, or from 50 to 150 percent, wherein these ranges include the endpoints. Alternatively, exposure times can be increased by 1 percent up to 100 percent by increasing the surfactant concentration of the NO topical foam formulation. Alternatively, exposure times can be increased by 1 percent up to 50 percent by increasing the surfactant concentration of the NO topical foam formulation.

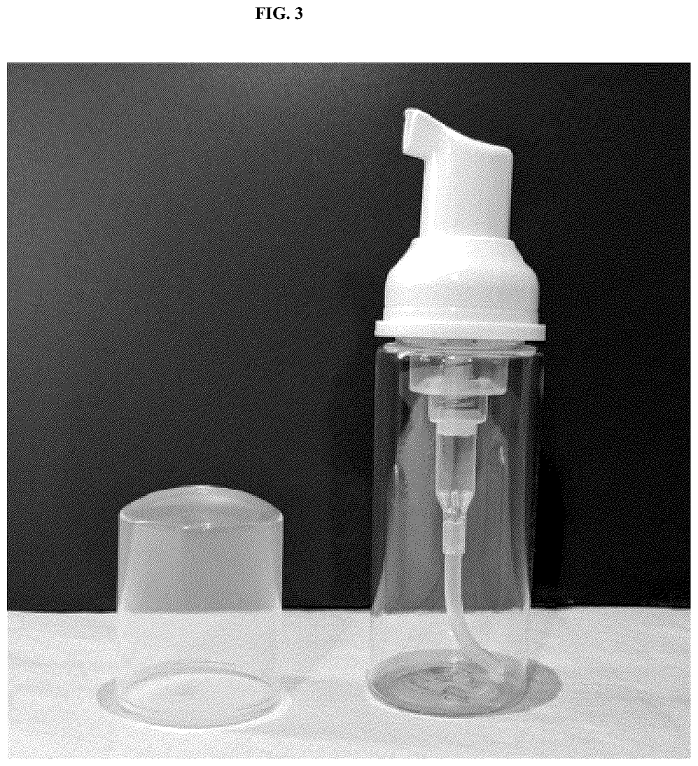

Alternatively, NO exposure times can be modulated by modifying the shear rate as the foam is administered. For example, shear rate can be adjusted by pumping the delivery device depicted in FIG. 3 slower or faster. Alternatively, the volume of the delivery device can be made smaller or larger, or the aperture can be made smaller or larger, to increase or decrease the velocity of the foam and thus increase the shear rate on the gas/liquid mixture as the foam exits the device.

Alternatively, NO exposure times can be modulated by modulating the temperature. For example, employing a lower temperature, for instance, by storing the device in a refrigerator or with a coolant, will lower the viscosity of the solutions, increase the shear rate of the gas/liquid mixture, and give rise to a more stable (longer lasting) foam.

Alternative methods for modifying NO exposure times may include modifying other parameters, such as the pH of the solutions used to generate the foam, the gas/liquid ratio, the addition of viscosity modifying agents, employing combinations of surfactants, nitrite salts, or acids, or the addition of other topical excipients.

The following use of the NO-containing foams and methods disclosed herein are intended to be non-limiting.

### Conditions Requiring Treatment: Skin Infections and Inflammatory Disorders of the Skin, and Other Conditions

Nitric oxide is involved in a range of biological processes, including vasodilation, platelet and leukocyte aggregation and adhesion, cell proliferation, endothelial layer permeability, scavenging of superoxide radicals, antimicrobial effects, immunomodulatory effects, and wound repair. The NO topical foam formulation of the present invention is suitable to regulate such processes by the provision of NO and the transport and delivery of the NO to the target sites. Thus, NO topical foam formulation of the present invention can be used to treat infections caused by bacteria, viruses, or fungi, as well as inflammations, injuries, skin conditions and/or blood vessel disorders in humans and animals. Blood vessel disorders include chronic inflammatory skin diseases, acne, eczema, neurodermatitis, psoriasis, scars, wrinkles, abrasions, burns, trauma, hematoma, androgenetic alopecia (male-pattern hair loss and female-pattern hair loss), blepharitis, Raynaud syndrome, bacterial skin & soft-tissue infections, leg lesion causes by peripheral vascular disease, chronic venous insufficiency and/or ischemic vasculitis.

As provided herein, the NO topical foam formulation of the present invention is applied to a site on the skin. The NO topical foam formulation of the present invention can induce vasodilation and increase blood flow and angiogenesis. In one embodiment, the NO topical foam formulation of the present invention is applied to site on the skin that is infected with a virus. In an embodiment, the viral infection is chicken pox, shingles, molluscum contagiosum, roseola, measles, fifth disease rash, or mononucleosis rash.

In one embodiment, the NO topical foam formulation of the present invention is applied to site on the skin that is infected with a fungus. In an embodiment, the fungal skin infection is a Candida (yeast) infection, ringworm of the body, ringworm of the scalp, jock itch, Tinea versicolor, athlete's foot, onychomytosis, or fungal skin rashes.

In one embodiment, the NO topical foam formulation of the present invention is applied to site on the skin that is infected with bacteria. In one embodiment, the bacteria is planktonic bacteria. In another embodiment, the bacteria is biofilm bacteria.

Bacterial skin infections develop when bacteria enter through hair follicles or through small breaks in the skin that results from scrapes, punctures, surgery, burns, sunburn, animal or insect bites, wounds, and pre-existing skin disorders. Bacterial infections of the skin are described, for instance, in the Merck Manual, and include carbuncles, echtyma, erythrasma, folliculitis, furuncles, impetigo, lymphadenitis, skin abscesses, cellulitis, erysipelas, lymphangitis, necrotizing skin infections, wound infections, staphylococcal scalded skin fever, scarlet fever, toxic shock syndrome. ABSSI infections include cellulitis/erysipelas, wound infections and major cutaneous abscesses. Other bacterial infections of the skin include impetigo and minor cutaneous abscesses, animal or human bites, necrotizing fasciitis, diabetic foot infection, burns, chronic wound infections, myonecrosis, and echthyma gangenosum.

Many types of bacteria can infect the skin. The most common are Staphylococcus aureus (methicillin resistant S. aureus) and Streptococcus.

In one embodiment, the present invention relates to a method of treating a bacterial infection on the skin of a patient. In one embodiment, the bacterial infection is caused by P. aeruginosa, A. baumannii, S. aureus, S. epidermidis, P. mirabilis, Propionibacterium acnes, (P. acnes), Cutibacterium acnes, Malassezia spp. Bacteroides spp., Fusobacterium spp., and Peptostreptococcus spp., or combinations thereof.

In another embodiment, the formulation can also be used to treat inflammatory skin diseases. An estimated 20-25% of the population is affected by chronic, non-communicable inflammatory skin diseases, including atopic dermatitis, psoriasis, urticaria, prurigo nodularis, lichen planus, hidradenitis suppurativa, alopecia areata, vitiliogo, uticaria, pemphigus, bullous pemphigoid, mucus membrane pemphigoid, epidermolysis bullosa acquista, cryopyrin associated periodic disorder, Schnitzier's syndrome, dermatomyositis, and systemic sclerosis. Chronic skin inflammation can also in some rare cases be caused by autoinflammatory diseases, or rheumatic diseases, such as cutaneous lupus erythematosus or dermatomyositis.

The formulation can also be used to treat other conditions, including neuropathy, diabetic neuropathy, and nerve damage.

### Biofilm Disruption

Biofilm-related infections affect skin and wound conditions, including chronic wounds caused by diabetes, burns, pressure injuries, venous or arterial ulcers, or surgical procedures. Infections are common complications of burns, with surface-associated communities of microbial pathogens (bacteria, viruses, or fungi) known as biofilms forming within human burn wounds within 10-24 hours of injury. The presence of biofilms in burns is problematic as biofilms are more resistant to antimicrobial agents than their planktonic counterparts, thus rendering conventional treatment strategies ineffective, with 75% of extensively burned patients dying as a consequence of severe infection.

Moreover, according to the National Institutes of Health, 65 percent of all hospital-acquired infections are due to microbes growing as biofilms, and 80 percent of chronic infections are linked to biofilms. Similarly, the Center for Disease Control estimates that hospital-acquired infections account for an estimated 1.7 million infections and 99,000 associated deaths each year in American hospitals alone. The high morbidity and mortality rate is due to biofilms being extremely difficult to control in medical settings.

Conventional therapies have proven inadequate in the treatment of many (if not most) chronic biofilm infections, due to the extraordinary tolerance of biofilms to available antimicrobial agents relative to their planktonic counterparts, and their ability to inhibit healing.

The development of antimicrobial burn creams was considered a major advance in the care of burn wound patients, yet infections of wounds remain the most common cause of morbidity and mortality among the 6.5 million people suffering from wounds in the United States alone, causing over 200,000 deaths annually. The most affected are people suffering from burn wounds, for which almost 61% of deaths are caused by infection. Treatment failure has been primarily attributed to the virulence factors produced by the principal wound pathogens Staphylococcus aureus and Pseudomonas aeruginosa as well as their ability to form biofilms in wounds, which are recalcitrant to antibiotic treatment and the host immune defense.

Successful treatment of biofilm infections will require the development of novel treatment strategies such as the use of the NO containing foam formulations disclosed herein.

Thus, in an embodiment, the formulation can be used to disrupt biofilms by forming a bubble barrier over the biofilm. More specifically, the formulation can be used to eradicate biofilms. In one embodiment, the biofilms are associated with a microorganism selected from the group consisting of Candida albicans, Coagulase-negative staphylococci, Enterococcus spp., Klebsiella pneumonia, Pseudomonas aeruginosa, and Staphylococcus aureus, or a combination thereof.

In one embodiment, the present invention relates to a method of treating a biofilm-associated skin infections on the skin of a patient, comprising treating the skin of the patient with the NO topical formulation disclosed herein under conditions that are effective to treat the acne.

In one embodiment, the present invention relates to a method of treating acne, comprising treating the skin of the patient with the NO topical formulation disclosed herein under conditions that are effective to treat the biofilm-associated skin infection.

In one embodiment, the present invention relates to a method of preventing a biofilm-associated skin infections on the skin of a patient, comprising treating the skin of the subject with the NO topical formulation disclosed herein under conditions that are effective to prevent the acne or biofilm-associated skin infection.

In one embodiment, the present invention relates to a method of preventing acne, comprising treating the skin of the patient with the NO topical formulation disclosed herein under conditions that are effective to prevent acne.

In one embodiment, the present invention relates to a method of inhibiting biofilm formation on the surface of the skin of a patient, comprising applying the NO topical formulation disclosed herein to the surface. As used herein, “inhibiting a biofilm” means that at least some of the microbial pathogens lodged in the biofilm matrix are killed, so that over time the biofilm will degrade over time.

In one embodiment, the present invention relates to a method of disrupting biofilm formation on the surface of the skin of a patient, comprising applying the NO topical formulation disclosed herein to the surface. As used herein, “disrupting or degrading a biofilm” means that at least some of the microbial pathogens lodged in the biofilm matrix are killed, so that over time the biofilm will degrade over time.

In one embodiment, the present invention relates to a method of inhibiting biofilm formation on the surface of the skin of a patient, comprising applying the NO topical formulation disclosed herein to the surface. In one embodiment, the surface is the skin of a patient requiring treatment. In a further embodiment, the biofilm contains P. aeruginosa, A. baumannii, S. aureus, S. epidermidis, C. albicans, S. epidermidis, P. mirabilis, Propionibacterium acnes, (P. acnes), Cutibacterium acnes, Malassezia spp., Bacteroides spp., Fusobacterium spp., and Peptostreptococcus spp., or combinations thereof.

In one embodiment, the present invention relates to a method of disrupting or degrading a biofilm formation on the surface of the skin of a patient, comprising applying the NO topical formulation disclosed herein to the surface. In one embodiment, the surface is the skin of a patient requiring treatment. In a further embodiment, the bacteria contained in the biofilm is selected from the group consisting of P. aeruginosa, A. baumannii, S. aureus, S. epidermidis, C. albicans, S. epidermidis, P. mirabilis, Propionibacterium acnes, (P. acnes), Cutibacterium acnes, Malassezia spp., Bacteroides spp., Fusobacterium spp., and Peptostreptococcus spp., or combinations thereof.

In one embodiment, the present invention relates to a method of killing a microbe contained in a biofilm on a surface, comprising applying the NO topical formulation disclosed herein to the surface. In one embodiment, the surface is the skin of a patient requiring treatment.

In one embodiment, the present invention relates to a method of disrupting or degrading a biofilm or killing bacteria contained in a biofilm on a surface, comprising applying the NO topical formulation disclosed herein to the surface. In one embodiment, the surface is the skin of a patient requiring treatment. In a further embodiment, the bacteria contained in the biofilm is selected from the group consisting of P. aeruginosa, A. baumannii, S. aureus, S. epidermidis, C. albicans, S. epidermidis, P. mirabilis, Propionibacterium acnes, (P. acnes), Cutibacterium acnes, Malassezia spp., Bacteroides spp., Fusobacterium spp., and Peptostreptococcus spp., or combinations thereof.

In another embodiment, the present invention relates to a method of killing bacteria contained in a biofilm on a surface, comprising applying the NO topical formulation disclosed herein to the surface. In one embodiment, the surface is the skin or tissue of a patient requiring treatment.

In another embodiment, the formulation can be combined with other skin infection and biofilm eradication/disruption treatments. Antibiotics such as fluoroquinolones such as ciprofloxacin, rifampin, and ampicillin are known to penetrate but incompletely eradicate biofilm bacteria. Thus, in a further embodiment, the formulation can be administered before, after, or at the same time (concurrently) as these other biofilm eradicating treatments for the skin are administered. Such treatments are commonly administered orally or intravenously.

Thus, the formulation can be administered prior to, concurrently with, or subsequent to treatment with an antibiotic that targets, Staph aureus, Strep pyogenes, Strep agalactiae, Staph dysgalctiae, Staph anginosos, Staph intermedius, Staph constellatus, Entero faecalis, E. coli, Klebisella pneumonia, Enterobacter cloacae, and Pseudomonas aeruginosa. In one embodiment, the formulation can be administered prior to, concurrently with, or subsequent to antibiotic treatment selected from the group consisting of dalbavancin, oritavancin, tedizolid, and delafloxacin, vancomycin, linezolid, clindamycin, daptomycin, ceftaroline, docycycline, minocycline, trimethorprim-sulfamethoxazole telavancin, omadacycline, and lefamulin.

In another embodiment, the method relates to a method of removing or loosening foreign material from a topical site on the skin, comprising applying the NO topical formulation disclosed herein to the site. The foreign material may be microbial, that is, bacterial, viral or fungal, as described herein, or contained within a biofilm, and infection-causing or otherwise irritating to the skin.

In another embodiment, the method relates to a method of reducing or removing bioburden from a topical site on the skin, comprising applying the NO topical formulation disclosed herein to the site.

In another embodiment, the method relates to a method of disinfecting a topical site on the skin, comprising applying the NO topical formulation disclosed herein to the site.

### List of Additional Embodiments

Embodiment 1. A method of treating a topical site on a patient requiring treatment, comprising contacting the site with a NO topical foam formulation comprising NO, water, and a surfactant, wherein the concentration of the surfactant is 0.1 weight/weight (w/w) to 50% w/w.

Embodiment 2. The method of embodiment 1, wherein the site is a skin wound, burn, infection, site of inflammation, or other skin condition requiring treatment.

Embodiment 3. The method of embodiments 1-2, wherein the surfactant is a cationic surfactant.

Embodiment 4. The method of embodiments 1 to 3, wherein the NO comes from a source of NO.

Embodiment 5. The method of embodiments 1-4 further comprising an acid.

Embodiment 6. The method of embodiments 1 to 5, wherein the formulations employed to make the NO topical foam formulation comprises two formulations, wherein the first formulation comprises water, acid, and the surfactant; and the second formulation comprises water, the surfactant, and a nitrite salt.

Embodiment 7. The method of embodiments 1 to 6, wherein the acid concentration (w/w %) of the first solution is 1 to 30 percent and the surfactant concentration of the first solution is 0.1 to 50 percent (w/w).

Embodiment 8. The method of embodiments 1 to 6, wherein the nitrite concentration (w/w %) of the second solution is 1 to 30 percent and the surfactant concentration of the second solution is 0.1 to 50 percent (w/w).

Embodiment 9. The method of embodiments 1-8, wherein:
the surfactant is selected from the group consisting of cetyl trimethyl ammonium bromide, cetrimonium bromide, dodecylbenzenesulfonic acid, cetylpyridinium chloride, stearalkonium chloride, polyquaternium-7, cocamidopropyl betaine, coco betaine, lauryl dimethyl ammonium chloride, polyquaternium-10, behentrimonium chloride, and cetrimonium chloride or combinations thereof,
the NO comes from a source comprising one or more of a NO gas, nitrite salt, NONOate, NO-polymer, nano-crystalline NO, NO-metal, NO silica particles, nitrate esters, nitroprusside, nitrosamine, L-arginine, L-citrulline, nitrosothiols, NO synthase or synthase upregulators; and
the acid is selected from the group consisting of citric acid, lactic acid, salicylic acid, phosphoric acid, ascorbic acid, hydrochloric acid, acetic acid, hyaluronic acid, and hypochlorous acid or combinations thereof.

Embodiment 10. The method of embodiment 9, wherein the NO comes from a source selected from the group consisting of sodium nitrite, calcium nitrite, potassium nitrite, and ammonium nitrite or combinations thereof.

Embodiment 11. The method of embodiment 10, wherein the surfactant is cocobetaine, the nitrite is sodium nitrite, and the acid is citric acid.

Embodiment 12. The method of embodiments 1 to 11, further comprising one or more additional topical dermatological agents.

Embodiment 13. The method of embodiments 1-12, wherein NO exposure times are increased by increasing the surfactant concentration, increasing the shear rate, r lowering the temperature of the NO topical foam formulation.

Embodiment 14. The method of embodiments 1-12, wherein NO exposure times are increased from 10 seconds to 72 hours by increasing the surfactant concentration, increasing the shear rate, or lowering the temperature of the NO topical foam formulation.

Embodiment 15. The method of embodiments 1-14, wherein the site is a skin infection selected from the group consisting of a bacterial infection, a viral infection, and a fungal infection, and wherein the skin infection exists on a wound, a burn, or site of another condition requiring treatment.

Embodiment 16. The method of embodiment 15, wherein the infection is a planktonic infection (for instance, by planktonic bacteria), or an infection caused in conjunction with a biofilm (for instance, by a microbe encased in a biofilm).

Embodiment 17. The method of embodiment 16, wherein the formulation of claims 1-13 is administered prior to, concurrent with, or subsequent to antibiotic treatment.

Embodiment 18. A method of treating, inhibiting, or eradicating an infection on a topical site of a patient requiring such treatment, comprising:
applying the NO topical foam formulation foam as recited in embodiments 1-13 to the topical site requiring treatment;
allowing the NO topical foam formulation to remain on the topical site for a period of time to allow the NO to treat the infection; and
allowing the NO topical foam formulation to remain on the topical site for a period of time to allow the NO to be absorbed and to provide a therapeutic effect;
wherein the infection is caused by a bacteria, a virus, or a fungus; and
wherein the therapeutic effect is an antimicrobial effect, vasodilatory effect, or healing effect.

Embodiment 19. A method of disrupting or degrading a biofilm on the topical site of a patient requiring such treatment and providing a therapeutic effect, comprising:
applying the NO topical foam formulation foam as recited in embodiments 1-13 to the topical site requiring treatment;
allowing the NO topical foam formulation to remain on the topical site for a period of time to allow the NO to disrupt or degrade the biofilm; and
allowing the NO topical foam formulation to remain on the topical site for a period of time to allow the NO to be absorbed and provide a therapeutic effect;
wherein the biofilm encases bacteria, a virus, or a fungus; and
wherein the therapeutic effect is an antimicrobial effect, vasodilatory effect, or wound healing effect.

Embodiment 20. A method of reducing the bioburden on the topical site of a patient requiring such treatment and providing a therapeutic effect, comprising:
applying the NO topical foam formulation foam as recited in embodiments 1-13 to the topical site; and
allowing the NO topical foam formulation to remain on the topical site for a period of time to allow the NO to reduce the bioburden.

Embodiment 21. A method of disinfecting a topical site of a human or animal requiring disinfection, comprising:
applying the NO topical foam formulation foam as recited in claims 1-13 to the topical site requiring disinfection
allowing the NO topical foam formulation to remain on the topical site for a period of time to allow the NO to exert its disinfecting effect.

Embodiment 22. A method of removing or loosening foreign material from a topical site, comprising:
applying the NO topical foam formulation foam as recited in embodiments 1-13 to the topical site;
allowing the NO topical foam formulation to remain on the topical site for a period of time to allow the NO to remove or loosen the foreign material; and
allowing the NO topical foam formulation to remain on the topical site for a period of time to allow the NO to be absorbed and to provide the removing or loosening effect.

Embodiment 23. A method of:
(i) treating, inhibiting, or eradicating an infection on a topical site of a patient requiring such treatment, or of
(ii) disrupting, degrading, or removing a biofilm on the topical site of a patient requiring such treatment, or of
(iii) reducing or removing the bioburden on the topical site of a patient requiring such treatment, or of
(iv) removing or loosening foreign material from a topical site; or of
(v) disinfecting a topical site of a human or animal comprising:
   applying the NO topical foam formulation foam as recited in claims 1-13 to the topical site;
   allowing the NO topical foam formulation to remain on the topical site for a period of time to allow the NO to treat inhibit, or eradicate the infection or to disrupt, degrade, or remove the biofilm, or to reduce or remove the bioburden, or to remove or loosen the foreign material, or to disinfect; or
   allowing the NO topical foam formulation to remain on the topical site for a period of time to allow the NO to be absorbed to provide a therapeutic effect;
   wherein the infection, biofilm, bioburden, is caused by a bacteria, a virus, or a fungus; or
   wherein the therapeutic effect is an antimicrobial effect, vasodilatory effect, or healing effect.

Embodiment 24. A method of:
(i) treating, inhibiting, or eradicating an infection on a topical site of a patient requiring such treatment, or of
(ii) disrupting, degrading, or removing a biofilm on the topical site of a patient requiring such treatment, or of
(iii) reducing or removing the bioburden on the topical site of a patient requiring such treatment, or of
(iv) removing or loosening foreign material from a topical site; or of
(v) disinfecting a topical site of a human or animal comprising:
   applying the NO topical foam formulation foam as recited in claims 1-13 to the topical site; and
   allowing the NO topical foam formulation to remain on the topical site for a period of time to allow the NO to be absorbed to provide a therapeutic effect;
   wherein the therapeutic effect is an antimicrobial effect, vasodilatory effect, or healing effect.

Embodiment 25. A method of:
(i) treating, inhibiting, or eradicating an infection on a topical site of a patient requiring such treatment, or of
(ii) disrupting, degrading, or removing a biofilm on the topical site of a patient requiring such treatment, or of
(iii) reducing or removing the bioburden on the topical site of a patient requiring such treatment, or of
(iv) removing or loosening foreign material from a topical site; or of
(v) disinfecting a topical site of a human or animal comprising:
   applying the NO topical foam formulation foam as recited in claims 1-13 to the topical site; and
   allowing the NO topical foam formulation to remain on the topical site for a period of time to allow the NO to treat, inhibit, or eradicate the infection or to disrupt, degrade, or remove the biofilm, or to reduce or remove the bioburden, or to remove or loosen the foreign material, or to disinfect;
   wherein the infection, biofilm, or bioburden is caused by a bacteria, a virus, or a fungus.

Embodiment 26. A method of prolonging a therapeutic effect of the NO topical foam formulation recited in embodiments 1-13, the method comprising increasing the surfactant concentration of the NO topical foam formulation and decreasing the rate of NO delivery, wherein the therapeutic effect is an antimicrobial effect, a vasodilatory effect, or a wound healing effect.

Embodiment 27. A therapeutic covering for an application site on the skin of a patient requiring such treatment, wherein the covering is the NO topical formulation foam as recited in embodiments 1-13, wherein the covering protects the application site on the skin or tissue from ambient interference and environmental contaminants such as air- or waterborne pathogens.

### EXAMPLES

The invention will now be demonstrated by the following non-limiting examples.

### Example 1: Acidified Nitrite Foam Microbial Action

Nitric oxide (NO) plays critical roles in the healing of acute skin wounds including stimulating vasodilation, angiogenesis, and broad antimicrobial activity. As a gas, it diffuses throughout wounds and into adjacent tissues. NO can be produced when acidic compounds are mixed with nitrite salts. A novel method to generate and deliver NO gas at the point-of-care was developed using acidified nitrite in a bubble foam and was tested in vitro using the well-established drip-flow biofilm model against six common microbial wound pathogens. A single 5-minute topical exposure of the NO bubble gas formulation generated a 5.8-log 10 reduction of mature biofilm of Pseudomonas aeruginosa, a 5.1-log 10 reduction of Staphylococcus aureus biofilm, a 4.0-log 10 reduction of Staphylococcus epidermidis biofilm, a 3.2-log 10 reduction of Proteus mirabilis biofilm, a 2.7-log 10 reduction of Acinetobacter baumannii biofilm, and a 1.5-log 10 reduction of Candida albicans biofilm. These results suggest that a single treatment with topical NO foam may be able to effectively reduce the bioburden including biofilm phenotypes.

### Introduction

Wound healing is hindered by excessive bioburden, and NO generated in wounded tissues plays a critical role in host defense and immune response acting as a cytotoxic agent against pathogens (Nathan CF, 1991) as well as inducing inflammation (Nussler AK, 1993). In addition, NO plays key roles in maintaining vascular homeostasis, regulating inflammation, and antimicrobial action (Malone-Povolny MJ, 2019).

As an antimicrobial molecule, NO has both nitrosative and oxidative mechanisms which eventually result in the production of dinitrogen trioxide (N2O3) and peroxynitrite (ONOO⁻) (Wink DA, 1998). Dinitrogen trioxide induces DNA deamination, while peroxynitrite causes membrane lipid peroxidation (Moller MN, 2007). NO-mediated inhibition of metabolic enzymes may also constitute an important mechanism of NO-induced cytosis. (Jones ML, 2010). These harmful processes are specific only to bacteria because eukaryotic cells have mechanisms to scavenge these reactive species to prevent damage (Fang, 2011).

Exogeneous NO can be used to reduce wound bioburden and speed wound healing. Local administration of NO gas has been found to be especially challenging due to its highly reactive nature when applied to the skin and simultaneously exposed to ambient air. Even so, external applications of NO have been done using acidified nitrite following the pathway shown in Equations 1 and 2 depicted above.

Sodium nitrite, as used above, can be easily incorporated into a lotion or gel for topical application. The typical acidic environment in a healing skin wound can generate the NO species from sodium nitrite, however the addition of acidic co-reactants, such as citric acid, more effectively drives the conversion of nitrite to NO gas (Dave RN, 2012).

Clinically, an acidified nitrite/citric acid gel producing NO gas was shown to improve healing of various types of chronic wounds with major bacterial bioburdens. Treatment of Buruli abscesses, which is a chronic lesion with contributing mycobacterial infection, stimulated a 56% decrease in average lesion size compared to a placebo group (Phillips R, 2004). In wounds with substantial infection by methicillin resistant Staphylococcus aureus (MRSA), NO gas treatment resulted in full eradication of MRSA within five days in 15 wounds across eight patients (Ormerod AD, 2011).

Certain advantages can be realized by applying the acidified nitrite as a foam. In this context, a foam is comprised of small bubbles of gas surrounded by thin films of liquid. One solution of acid, surfactant, and water can be added to a hand pump that creates a foam when depressed. Another solution of nitrite salt, surfactant, and water can be added to another hand pump that creates a foam when depressed. When the two foams are mixed, gaseous NO is created in the liquid film. Similar to the case of the acidified nitrite gels and creams, the NO must move to the liquid/gas boundary. The thin film and the low viscosity of the film allows the NO gas to move quickly to the gas phase. Once the NO is in the gas phase, the bubbles of the foam keep the NO gas from escaping.

By reducing the mass transfer barrier that is present in the gel or cream formulations, another independent factor affecting the NO release profile can be manipulated. Specifically, the release rate of NO gas is directly related to the amount of mixing between the acid containing foam and the nitrite containing foam. By removing the rate limiting step of NO diffusing through a high viscosity gel or cream, the mixing of the two foams along with the concentration of acid and nitrite ions will directly affect the release profile of the NO gas.

The mixed NO producing foam will completely fill a wound and ensure intimate contact with any complicated surface topology due to the presence of the surfactant in the formulation. As the bubbles in the foam contact a surface, they will rupture due to temperature gradients and surface interactions thus releasing the NO against the surface (see FIG. 1).

To determine if acidified nitrite foam is as effective as gels and creams at killing microbial pathogens, a series of experiments were conducted by the Medical Biofilms Laboratory (MBL) of the Center for Biofilm Engineering (CBE) at Montana State University. Acidified nitrite foam was applied to biofilms created by six different microbes.

### Test Methods

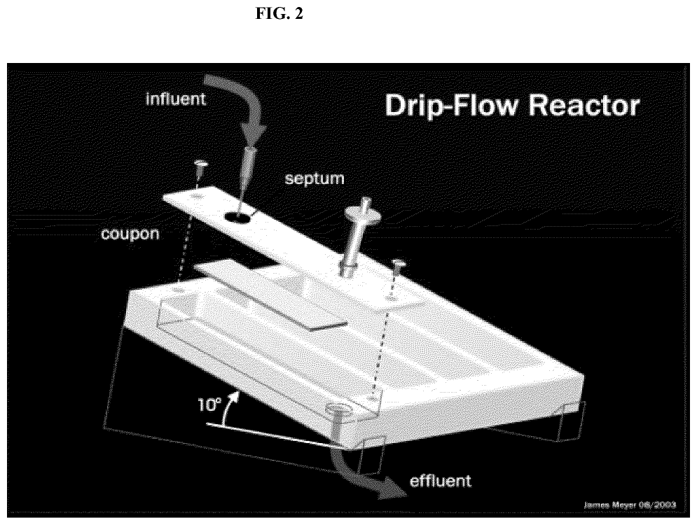

The testing was conducted by the Medical Biofilms Laboratory (MBL) of the Center for Biofilm Engineering (CBE) at Montana State University. Biofilms were created using an in-vitro model system, the Drip Flow Reactor (DFR 110-6, Biosurface Technologies Corp., Bozeman, MT). The DFR (FIG. 2) is designed to model a low shear environment and has been approved by ASTM as a standard method for growing P. aeruginosa biofilms (method E2647-20). Testing reported herein used a modified procedure, more relevant to wound biofilms.

### Pathogens

Testing was performed on biofilms of Pseudomonas aeruginosa MBL Strain SWR 215 (a clinical chronic wound isolate), Acinetobacter baumannii (ATCC BAA-1797), Staphylococcus aureus (ATCC BAA-1556), Candida albicans (ATCC 10231), Staphylococcus epidermidis (ATCC 35984), and Proteus mirabilis (ATCC 7002). These strains are maintained as a frozen stock culture at −80° C. in the MBL.

### Treatments

NOxy Health Products supplied the NO producing foam. The foam was supplied in two components (a solution A and a solution B) contained in foam pumps (see FIG. 3). Solution A comprised a 9.7% w/w solution of citric acid. Solution B comprised an 13% w/w solution of sodium nitrite.

The steps to apply an NO foam treatment are listed below.
1 Dispense an amount of Solution A foam into an appropriate container by depressing the Solution A pump head consistently and briskly;
2 Dispense an amount of Solution B foam into the same container by depressing the Solution B pump head consistently and briskly;
3 Using a 2.5-inch plastic paddle or similar tool, stir the two foams for five seconds to mix; and
4 Apply the mixed NO foam by pouring the foam out of the container where needed or using a spatula/spoon for more measured/precise placement of the foam.

### Biofilm Growth

Six-channel DFRs, equipped with hydroxyapatite- and collagen-coated (HAC) glass coupons, were operated at 33° C. (approximate wound temperature) under aerobic conditions. Hydroxyapatite-coated glass slides, prepared by Clarkson Chromatography, were collagen-coated using a coating matrix kit (Life Technologies Corporation) following the manufacturer's instructions.

Approximately 20 minutes prior to inoculation, sterile media (1%-strength brain-heart infusion broth with 0.5% adult bovine serum) was dripped into each channel and allowed to collect over the coupons. A conditioning layer on the surface of the coupons formed. Each channel of the reactor was then inoculated with 1 mL of an overnight culture of the test organism. The reactor was then set at a 100 angle and sterile media was dripped through the reactor at a rate of 10 mL/hour per channel for 72 hours.

### Biofilm Treatment and Sampling

For treatment, flow to the DFR was halted and the mixed NO foam or individual A and B foams were applied as directed to a coupon. Enough of the foam was applied to completely cover the entire DFR coupon (approximately 20 mL of foam). Before applying the foams, the untreated control coupon was removed from the DFR and analyzed by plate count, as described below.

Following the contact time, the coupon(s) were removed from the DFR and rinsed with phosphate-buffered saline (PBS) to remove residual foam and unattached bacteria.

### Viable Plate Counts

The number of viable bacteria on the coupons was determined by viable plate count. After rinsing, the coupons were placed in 10 mL of 2×-strength Dey-Engley (DE) neutralization broth. Biofilm on the coupons were scraped and rinsed with the DE, then sonicated (30 sec), vortexed (2 min), and sonicated (30 sec) to further remove and disaggregate the biofilms. The biofilm suspensions were then serially diluted with PBS and plated on Tryptic Soy Agar (TSA). The plates were incubated at 37° C. for 24-48 hours and then the number of Colony-Forming Units (CFU) counted. Based on the dilution and surface area of the slide, the number of CFU per unit area was calculated and logarithmically (base 10) transformed. Log differences between the treatments and untreated control biofilms was calculated for each experiment.

### Results and Analysis

For each test organism, the viable plate counts in CFU/cm² for an untreated control (no treatment), a Solution A foam treatment, a Solution B foam treatment, and three NO foam mixture treatments were measured. These six measurements corresponded to the six plates in the DFR. All foam treatments lasted five minutes. Each CFU/cm² measurement is reported in the following Tables.

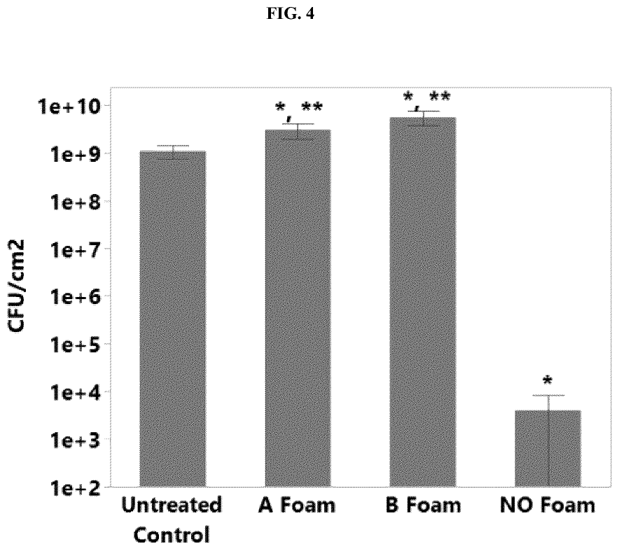

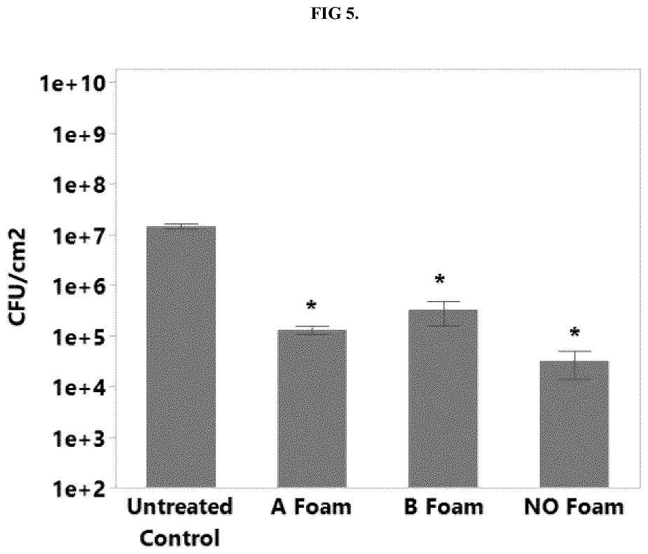

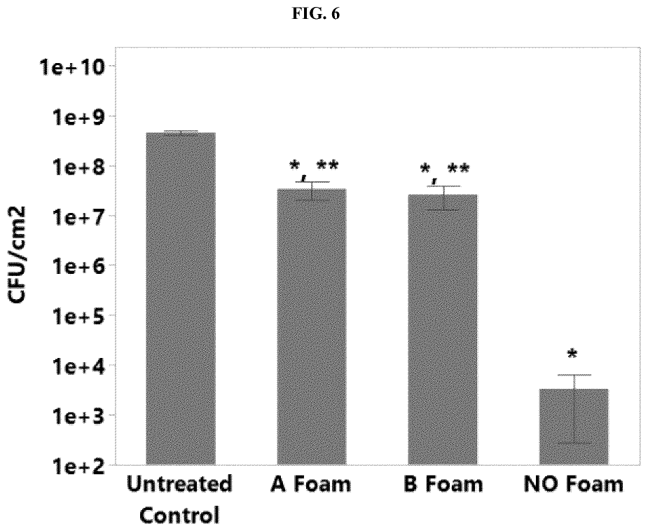

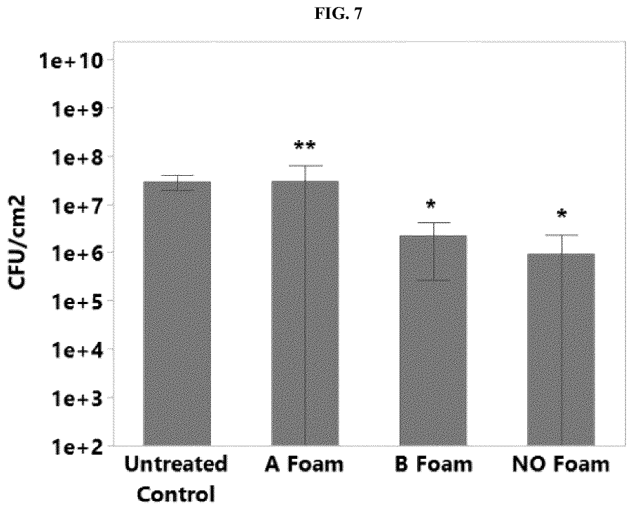

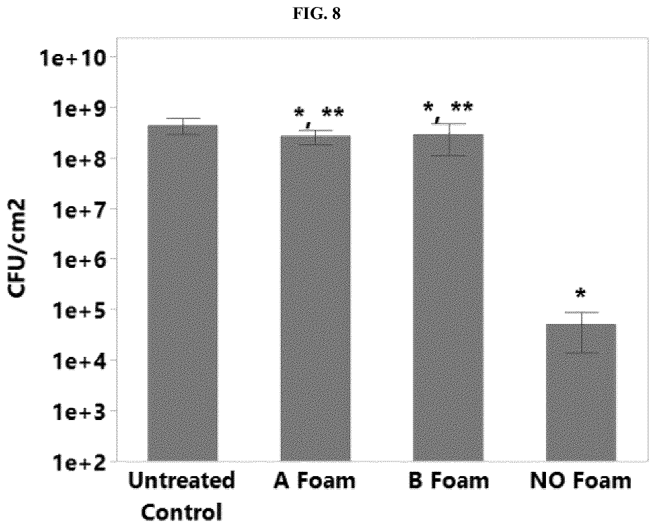

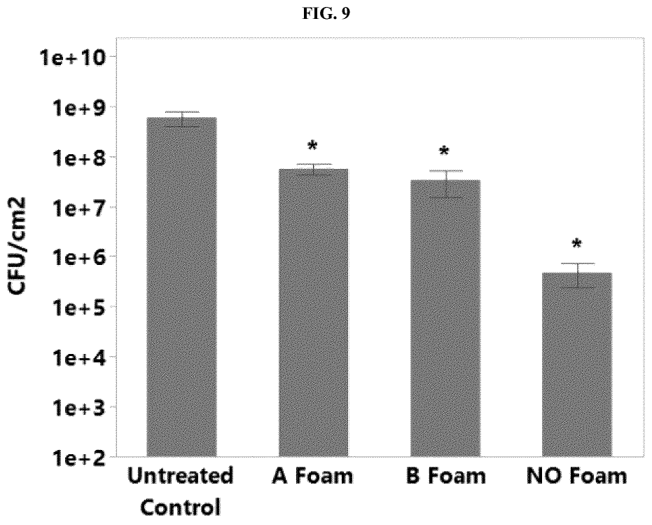

Tables 1.1-1.6 and FIGS. 4-9 summarize the results.

Table 1 summarizes the average log reduction of between the control and the five-minute NO foam treatment. Since a five-minute treatment of NO foam was significant for all organisms tested, Table 1.1 also lists the probability that the difference is not significant.

TABLE 1.1
Microbe Log Reduction Probability

| Microbe | Log Reduction | Probability |
|---|---|---|
| P. aeruginosa | 5.8 | <0.0004 |
| A. baumannii | 2.7 | <0.0001 |
| S. aureus | 5.1 | <0.0001 |
| C. albicans | 1.5 | <0.0001 |
| S. epidermidis | 4.0 | <0.0001 |
| P. mirabilis | 3.2 | <0.0001 |

CFU/cm² measurements are reported in Tables 1.2-1.6 for each bacteria tested, along with accompanying FIGS. 4-9.

TABLE 1.2
Log Reduction Summary of All Tests Using P. aeruginosa

| Sample | Log CFU/cm² | Log Reduction |
|---|---|---|
| Untreated Control | 8.97 | |
| Untreated Control | 9.16 | |
| Untreated Control | 8.93 | |

Five Minute Treatment

| | | |
|---|---|---|
| A Foam | 9.48 | −0.54 |
| B Foam | 9.74 | −0.80 |
| NO Foam | 3.96 | 5.01 |
| NO Foam | 2.78 | 6.37 |
| NO Foam | 3.66 | 5.49 |
| NO Foam | 2.81 | 6.34 |

TABLE 1.3
Log Reduction Summary of All Tests Using A. baumannii

| Sample | Log CFU/cm² | Log Reduction |
|---|---|---|
| Untreated control | 7.16 | |
| Untreated control | 7.64 | |

Five Minute Treatment

| | | |
|---|---|---|
| A Foam | 5.11 | 2.05 |
| A Foam | 5.16 | 2.48 |
| B Foam | 5.51 | 1.65 |
| B Foam | 7.51 | 0.14 |
| NO Foam | 4.33 | 2.83 |
| NO Foam | 4.31 | 2.85 |
| NO Foam | 4.72 | 2.44 |
| NO Foam | 5.17 | 2.47 |
| NO Foam | 4.69 | 2.95 |
| NO Foam | 3.69 | 3.95 |

TABLE 1.4
Log Reduction Summary of All Tests Using S. aureus tests

| Sample | Log CFU/cm² | Log Reduction |
|---|---|---|
| Untreated control | 8.65 | |

Five Minute Treatment

| | | |
|---|---|---|
| A Foam | 7.52 | 1.13 |
| B Foam | 7.41 | 1.24 |
| NO Foam | 3.04 | 5.61 |
| NO Foam | 3.74 | 4.91 |
| NO Foam | 5.91* | 2.74* |

The results from the NO Foam sample in the last row were excluded because the NO foam used to treat the three NO Foam plates was mixed at one time instead of using freshly mixed foam for each plate.

TABLE 1.5
Log Reduction Summary of All Tests Using C. albicans

| Sample | Log CFU/cm² | Log Reduction |
|---|---|---|
| Untreated control | 7.48 | |

Five Minute Treatment

| | | |
|---|---|---|
| A Foam | 6.37 | 1.10 |
| A Foam | 7.76 | −0.30 |
| B Foam | 5.73 | 1.75 |
| B Foam | 6.60 | 0.86 |
| NO Foam | 5.56 | 1.92 |
| NO Foam | 6.51 | 0.97 |
| NO Foam | 6.20 | 1.27 |
| NO Foam | 4.93 | 2.53 |
| NO Foam | 4.97 | 2.49 |
| NO Foam | 5.55 | 1.91 |

TABLE 1.6
Log Reduction Summary of All Tests Using S. epidermidis

| Sample | Log CFU/cm² | Log Reduction |
|---|---|---|
| Untreated control | 8.64 | |

Five Minute Treatment

| | | |
|---|---|---|
| A Foam | 8.43 | 0.21 |
| B Foam | 8.46 | 0.18 |
| NO Foam | 4.46 | 4.18 |
| NO Foam | 4.90 | 3.74 |
| NO Foam | 4.65 | 3.99 |

TABLE 1.7
Log Reduction Summary of All Tests Using P. mirabilis

| Sample | Log CFU/cm² | Log Reduction |
|---|---|---|
| Untreated control | 8.77 | |

Five Minute Treatment

| | | |
|---|---|---|
| A Foam | 7.74 | 1.02 |
| B Foam | 7.52 | 1.25 |
| NO Foam | 5.85 | 2.92 |
| NO Foam | 5.69 | 3.08 |
| NO Foam | 5.33 | 3.44 |

To determine if the differences in the CFU/cm² before and after the various treatments were statistically significant, an ANOVA analysis was conducted using the Tukey Honest Statistic Difference (HSD) test on JMP 16.1 (SAS Institute Inc.). FIGS. 4 through 9 show the control and each treatment CFU/cm² for each organism tested.

Acidified nitrite foam significantly reduced the CFU/cm² of each microbe tested after five minutes of treatment. The P. aeruginosa, S. aureus, S. epidermidis, and P. mirabilis seem particularly susceptible to NO antimicrobial actions. The A. baumannii seems less susceptible, and the C. albicans seems the least vulnerable consistent with the large volume of results of acidified nitrite gels/creams and gaseous NO in general.

### CONCLUSIONS

Acidified nitrite foam is an effective antimicrobial agent against several tested biofilms. To date, P. aeruginosa, A. baumannii, S. aureus, C. albicans, and S. epidermidis biofilms have been tested against a five-minute treatment of acidified nitrite foam. All the microbes experienced a significant log reduction in CFU/cm².

Historically, some antimicrobial treatments have performed well in a laboratory setting but perform less well against biofilms in vivo. It is thought that the biofilm penetrates the substrate it is growing on giving the biofilm extra protection from the antimicrobial agent. In other cases, the in vivo environment may neutralize the antimicrobial agent reducing its effectiveness against the biofilm pathogen.

Given the advantages of the acidified nitrite foam system and its effectiveness detailed here, it is hoped that acidified nitrite will become a preferred treatment in the clinician's toolbox to fight infection.

### REFERENCES

Dave R N, J. H. (2012). Biomedical evaluation of a novel nitrogen oxides releasing wound dressing. J Mater Sci Mater Med, 23(12), 3097-106.
Fang, F. (2011). Anitmicrobial actions of reactive oxygen species. mBio, 2(5), e00141-11.
Jones M L, G. J. (2010). Anitmirobial properties of NO and its application in antimicrobial formulations and medical devices. Appl Microbiol Biotechnol, 88, 401-7.
Malone-Povolny M J, M. S. (2019). Nitric Oxide Therapy for Diabetic Wound Healing. Adv Healthc Mater, 8(12), e1801210.
Moller M N, L. Q. (2007). Acceleration of NO autooxidation and nitrosation by membranes. IUBMB Life, 59(4-5), 243-8.
Nathan C F, H. J. (1991). Role of NO synthesis in macrophage antimicrobial activity. Curr Opin Immunol, 3(1), 65-70.
Nussler A K, B. T. (1993). Inflammation, immunoregulation, and inducible NO synthase. J Leukoc Biol, 54(2), 171-8.
Ormerod A D, S. A. (2011). An observational prospective study of topical acidified nitrite for killing methicillin-resistant Staphylococcus aureus (MRSA) in contaminated wounds. BMC Res Notes, 4, 458.
Phillips R, A. O.-J. (2004). Pilot randomized double-blind trial of treatment of Mycobacterium lesionans disease (Buruli lesion) with topical nitrogen oxides. Antimicrob Agents Chemother, 48(8), 2866-70.
Wink D A, M. J. (1998). Chemical biology of NO: Insights into regulatory, cytotoxic, and cytoprotective mechanisms of NO. Free Radic Biol Med, 25(4-5), 434-56.

### Example 2: Effect of Surfactant Concentration on NOx Production

### Summary

The production of NOx in mixed acidified nitrite foam was measured for solutions of varying surfactant concentrations thus providing solutions of differing viscosities. We found that with the higher concentration of surfactant, the higher the viscosity, and the lower the total and maximum NOx measured over five minutes.

### Objective of Test and Tests Performed

To measure the effect of changing the concentration of surfactant in the formulation while keeping the concentration of acid and nitrite the same.

The measured responses to the change were the total NOx evolved in five minutes, the maximum NOx evolved in five minutes, and the shape of the NO evolution curves.

### Test Methods

Three different A and B solutions sets were created according to the amounts shown in Tables 1-3. The Thermo Scientific™ 42iQLS Low Source NO—NO₂—NO_{X} Analyzer was warmed up for 30 minutes before testing commenced. The data logging program was started with the filename YYM DD [Solution] Test [number].

TABLE 2.1
Formulation A1

| Formulation | Mass (g) | Conc. (w/w %) |
|---|---|---|
| Solution A | | |
| Deionized Water | 54.20 | 87.42% |
| Cationic Surfactant | 1.80 | 2.90% |
| Acid | 6.00 | 9.68% |
| Solution B | | |
| Deionized Water | 51.40 | 82.90% |
| Cationic Surfactant | 0.60 | 0.97% |
| Nitrite | 10.00 | 16.13% |

TABLE 2.2
Formulation A2

| Formulation | Mass (g) | Conc. (w/w %) |
|---|---|---|
| Solution A | | |
| Deionized Water | 100.00 | 84.51% |
| Cationic Surfactant | 6.87 | 5.81% |
| Acid | 11.45 | 9.68% |
| Solution B | | |
| Deionized Water | 100.00 | 81.93% |
| Cationic Surfactant | 2.36 | 1.94% |
| Nitrite | 19.69 | 16.13% |

TABLE 2.3
Formulation A3

| Formulation | Mass (g) | Conc. (w/w %) |
|---|---|---|
| Solution A | | |
| Deionized Water | 100.00 | 81.61% |
| Cationic Surfactant | 10.67 | 8.71% |
| Citric Acid | 11.86 | 9.68% |
| Solution B | | |
| Water | 100.00 | 80.97% |
| Cationic Surfactant | 3.59 | 2.90% |
| Nitrite | 19.92 | 16.13% |

### Procedure

Solutions A and B were each prepared in a pump bottle. The bottles were shaken to produce a foam. One pump of Solution A foam and one pump of Solution B foam were placed in a weigh boat. The contents of the weigh boat were then mixed for 5 seconds manually using a finger to stir the contents of the weigh boat. The weigh boat was then placed into the sampling bin of the 42iQLS analyzer, and the lid placed on top. The 42iQLS analyzer began measuring NOₓ released from the mixed foam in the weigh boat as indicated by the rise of both the NO and NO₂ measurements on the screen of the analyzer. Data collection stopped after 700 seconds of data, which provided 70 individual time points, and (i) the data logging program was halted; (ii) the weigh boat was removed from the bin and disposed of in a separate room; (iii) a fan was used to blow fresh air into the testing chamber for 10 seconds; and a timer was set for 20 minutes.

The next test was conducted when the analyzer measured less than 5 ppm total NOx.

Tests for each of A1, A2, and A3 were conducted in triplicate. Statistical significance was determined by comparing the means of the three groups using the Tukey Honest Statistical Difference (HSD) test.

### Results and Analysis

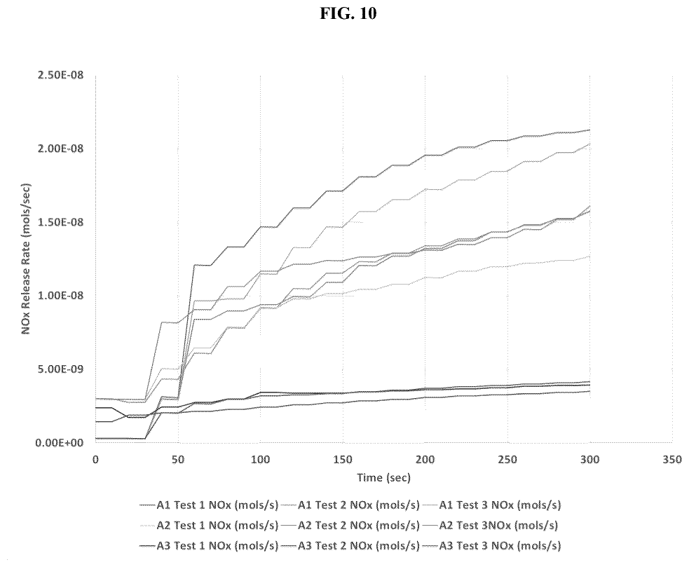

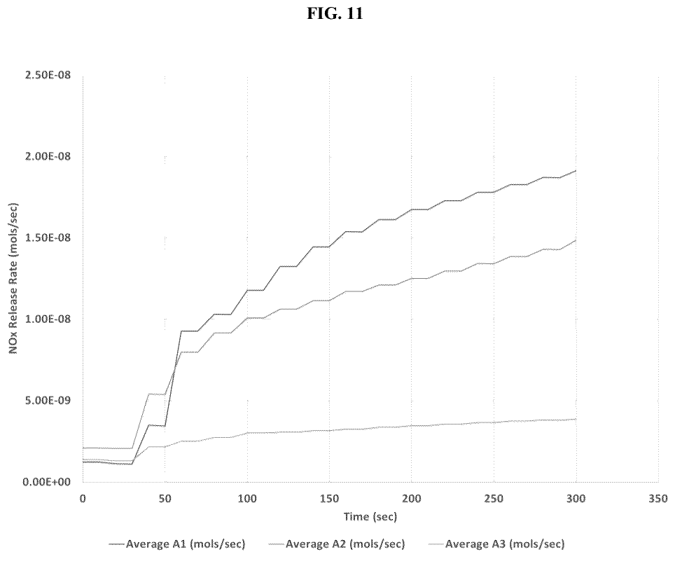

Table 2.4 summarizes the results. FIGS. 10 and 11 rates NOx release rates for the three foams containing different concentrations of surfactant. The results indicate that as the concentration of the cationic surfactant is increased, maximum NO release rate is decreased, and the total amount of NO released in 20 minutes is decreased. Thus, as surfactant concentration is increased, the total time over which the NO is released is longer, in accordance with conservation of mass principles.

TABLE 2.4

| | Surfactant | | Avg. | NOx Max | NOx Tot |
|---|---|---|---|---|---|
| Set | Concentration (%) | Test | Temperature | (mols/sec) | (mols) |
| A1 | 1.94 | 1 | 24.5 | 2.13E−08 | 4.53E−06 |
| A1 | 1.94 | 2 | 25.6 | 1.57E−08 | 3.19E−06 |
| A1 | 1.94 | 3 | 26.1 | 2.03E−08 | 3.94E−06 |
| A2 | 3.87 | 1 | 26.7 | 1.27E−08 | 2.82E−06 |
| A2 | 3.87 | 2 | 27.3 | 1.61E−08 | 3.47E−06 |
| A2 | 3.87 | 3 | 28.1 | 1.58E−08 | 3.10E−06 |
| A3 | 5.81 | 1 | 28.7 | 3.92E−09 | 9.97E−07 |
| A3 | 5.81 | 2 | 29.2 | 3.49E−09 | 8.25E−07 |
| A3 | 5.81 | 3 | 29.7 | 4.16E−09 | 9.32E−07 |

### Discussion and Conclusions

The results indicate that there is an optimal surfactant concentration to deliver NO gas for a given desired amount of time. The higher the concentration of surfactant in the formulation, the lower the maximum NOx. While the inverse relationships are not statistically significant, the results show that the more viscous the formulation (as a result of surfactant concentration), the lower the release rate of NOx.

### Example 3: Effect of Surfactant Concentration on Foam Collapse

### Summary

There is an optimal concentration of surfactant in a formulation that results in the longest time to foam collapse. This optimal concentration keeps the NO (NO) gas localized on the wound bed for the longest possible time for optimal wound healing and/or antimicrobial activity.

### Objective of Test and Tests Performed

The objective of the test was to measure the time it took for equal volumes of foam to collapse 50%. The concentration of the surfactant in three different foams were tested (see Tables 2.1-2.2).

### Test Methods

Several pumps of Solution A (five or six depending on the style of pump) and an equal number of pumps of Solution B were added to a polypropylene graduated cylinder. The foams were mixed with a polyvinyl rod for five seconds after which a stopwatch was started.

The stopwatch was stopped once the volume of the foam was reduced by 50%, and the time was recorded. The test was repeated two more times for each surfactant concentration.

Three different concentrations were tested (see Tables 2.1 and 2.3). Zero surfactant, A-1 surfactant, and A-3 surfactant concentration.

### Results and Analysis

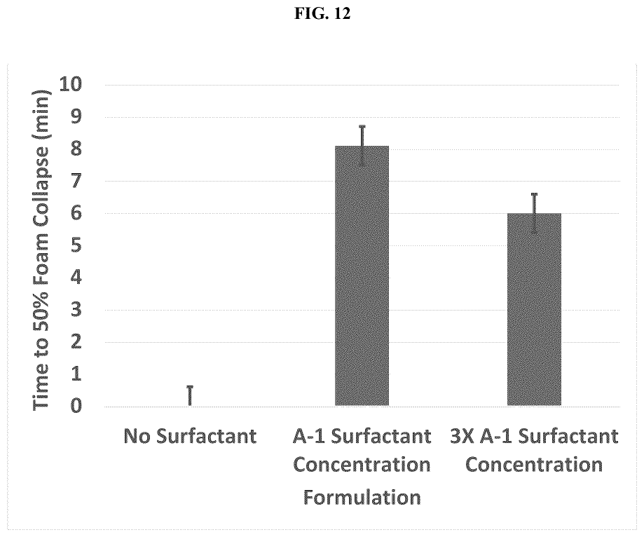

The time to 50% collapse for each concentration is shown in FIG. 12. The time went through a maximum at the A-1 concentration and dropped off at zero surfactant concentration and higher (A-3) concentrations. These results indicate that the amount of time over which the bubbles deliver their NO payload can be adjusted by adjusting the surfactant concentration. There is a maximum amount of time over which the NO can be delivered. Statistical significance was determined by comparing the means of the three groups using the Tukey Honest Statistical Difference (HSD) test.

### OTHER EMBODIMENTS

The foregoing disclosure has been described in some detail by way of illustration and example, for purposes of clarity and understanding. The invention has been described with reference to various specific and preferred embodiments and techniques. However, it should be understood that many variations and modifications can be made while remaining within the spirit and scope of the invention. It will be obvious to one of skill in the art that changes and modifications can be practiced within the scope of the appended claims. Therefore, it is to be understood that the above description is intended to be illustrative and not restrictive.

The scope of the invention should, therefore, be determined not with reference to the above description, but should instead be determined with reference to the following appended claims, along with the full scope of equivalents to which such claims are entitled.

1. A method of treating a topical site on a patient requiring treatment, comprising contacting the site with a NO topical foam formulation comprising NO, water, and a surfactant, wherein the concentration of the surfactant is 0.1 weight/weight (w/w) to 50% w/w.

2. The method of claim 1, wherein the site is a skin wound, burn, infection, site of inflammation, or other skin condition requiring treatment.

3. The method of claims 1-2, wherein the surfactant is a cationic surfactant and the NO comes from a source of NO.

4. The method of claims 1-3 further comprising an acid.

5. The method of claims 1 to 4, wherein the formulations employed to make the NO topical foam formulation comprises two formulations, wherein the first formulation comprises water, acid, and the surfactant; and the second formulation comprises water, the surfactant, and a nitrite salt.

6. The method of claims 1 to 5, wherein the acid concentration (w/w %) of the first solution is 1 to 30 percent and the surfactant concentration of the first solution is 0.1 to 50 percent (w/w).

7. The method of claims 1 to 5, wherein the nitrite concentration (w/w %) of the second solution is 1 to 30 percent and the surfactant concentration of the second solution is 0.1 to 50 percent (w/w).

8. The method of claims 1-7, wherein:
the surfactant is selected from the group consisting of cetyl trimethyl ammonium bromide, cetrimonium bromide, dodecylbenzenesulfonic acid, cetylpyridinium chloride, stearalkonium chloride, polyquaternium-7, cocamidopropyl betaine, coco betaine, lauryl dimethyl ammonium chloride, polyquaternium-10, behentrimonium chloride, and cetrimonium chloride or combinations thereof,
the NO comes from a source comprising one or more of a NO gas, nitrite salt, NONOate, NO-polymer, nano-crystalline NO, NO-metal, NO silica particles, nitrate esters, nitroprusside, nitrosamine, L-arginine, L-citrulline, nitrosothiols, NO synthase or synthase upregulators; and
the acid is selected from the group consisting of citric acid, lactic acid, salicylic acid, phosphoric acid, ascorbic acid, hydrochloric acid, acetic acid, hyaluronic acid, and hypochlorous acid or combinations thereof.

9. The method of claim 8, wherein the NO comes from a source selected from the group consisting of sodium nitrite, calcium nitrite, potassium nitrite, and ammonium nitrite or combinations thereof.

10. The method of claim 9, wherein the surfactant is cocobetaine, the nitrite is sodium nitrite, and the acid is citric acid.

11. The method of claims 1 to 10, further comprising one or more additional topical dermatological agents.

12. The method of claims 1-11, wherein the rate of NO release is decreased by increasing the surfactant concentration, increasing the shear rate, or lowering the temperature.

13. The method of claims 1-11, wherein NO exposure times are increased from 10 seconds to 72 hours by increasing the surfactant concentration, increasing the shear rate, r lowering the temperature of the NO topical foam formulation.

14. The method of claims 1-13, wherein the site is a skin infection selected from the group consisting of a bacterial infection, a viral infection, and a fungal infection, and wherein the skin infection exists on a wound, a burn, or site of another condition requiring treatment.

15. The method of claim 14, wherein the infection is a planktonic infection, or an infection caused in conjunction with a biofilm.

16. The method of claim 15, wherein the formulation of claims 1-12 is administered prior to, concurrent with, or subsequent to antibiotic treatment.

17. A method of:
(i) treating, inhibiting, or eradicating an infection on a topical site of a patient requiring such treatment, or of
(ii) disrupting, degrading, or removing a biofilm on the topical site of a patient requiring such treatment, or of
(iii) reducing or removing the bioburden on the topical site of a patient requiring such treatment, or of
(iv) removing or loosening foreign material from a topical site; or of
(v) disinfecting a topical site of a human or animal comprising:
applying the NO topical foam formulation foam as recited in claims 1-13 to the topical site; and
allowing the NO topical foam formulation to remain on the topical site for a period of time to allow the NO to be absorbed to provide a therapeutic effect;
wherein the therapeutic effect is an antimicrobial effect, vasodilatory effect, or healing effect.

18. A method of:
(i) treating, inhibiting, or eradicating an infection on a topical site of a patient requiring such treatment, or of
(ii) disrupting, degrading, or removing a biofilm on the topical site of a patient requiring such treatment, or of
(iii) reducing or removing the bioburden on the topical site of a patient requiring such treatment, or of
(iv) removing or loosening foreign material from a topical site; or of
(v) disinfecting a topical site of a human or animal comprising:
applying the NO topical foam formulation foam as recited in claims 1-13 to the topical site; and
allowing the NO topical foam formulation to remain on the topical site for a period of time to allow the NO to treat, inhibit, or eradicate the infection or to disrupt, degrade, or remove the biofilm, or to reduce or remove the bioburden, or to remove or loosen the foreign material, or to disinfect;
wherein the infection, biofilm, or bioburden is caused by a bacteria, a virus, or a fungus.

19. A method of prolonging a therapeutic effect of the NO topical foam formulation recited in claims 1-13, the method comprising increasing the surfactant concentration of the NO topical foam formulation and decreasing the rate of NO delivery, wherein the therapeutic effect is an antimicrobial effect, a vasodilatory effect, or a wound healing effect.

20. A therapeutic covering for an application site on the skin of a patient requiring such treatment, wherein the covering is the NO topical formulation foam as recited in claims 1-13, wherein the covering protects the application site on the skin or tissue from ambient interference and environmental contaminants such as air- or waterborne pathogens.
